(19) United States
(12) Patent Application Publication
(10) Pub. No.: US 2025/0382675 A1
(43) Pub. Date: Dec. 18, 2025

(54) COMPOSITION FOR DIAGNOSING PERIODONTAL DISEASE USING BACTERIAL POPULATION IN SALIVA, AND USE THEREOF

(71) Applicant: AJOU UNIVERSITY INDUSTRY-ACADEMIC COOPERATION FOUNDATION, Suwon-si, Gyeonggi-do (KR)

(72) Inventors: Suk JI, Seoul (KR); Joong Ki KOOK, Gwangju (KR); Soon Nang PARK, Gwangju (KR); Yun Kyong LIM, Gwangju (KR); Jae-Suk JUNG, Suwon-si, Gyeonggi-do (KR); Geum Hee CHOI, Gunpo-si, Gyeonggi-do (KR)

(73) Assignee: AJOU UNIVERSITY INDUSTRY-ACADEMIC COOPERATION FOUNDATION, Suwon-si, Gyeonggi-do (KR)

(21) Appl. No.: 18/700,295

(22) PCT Filed: Oct. 18, 2022
(86) PCT No.: PCT/KR2022/015871

§ 371 Date: Apr. 11, 2024

(30) Foreign Application Priority Data
Oct. 19, 2021 (KR) 10-2021-0138897

(51) Int. Cl. C12Q 1/689

(52) U.S. Cl. CPC C12Q 1/689; C12Q 2600/158

(57) ABSTRACT

The present invention relates to a composition for diagnosing a periodontal disease using the bacterial population in saliva, and a use thereof. The purpose of the present invention is to determine the severity of periodontal disease by using the population and relative ratio of bacteria distributed in saliva, wherein differences in bacterial population characteristics are exhibited according to whether the periodontal disease is gingivitis, moderate periodontitis, or severe periodontitis, and the present invention presents criteria for determining the severity of periodontal disease by using the differences, and in addition, with respect to sampling, saliva is safe, can be accessed easily and quickly, and can be sampled non-invasively to minimize the inconvenience to a patient, and the development of a diagnostic kit using saliva can help patients recognize for themselves the progression of periodontal disease.

### TECHNICAL FIELD

The present disclosure relates to a composition for diagnosing a periodontal disease using bacterial colonies in saliva and a use thereof.

### BACKGROUND ART

A periodontal disease is a chronic disease in which inflammation progresses with an unawareness of the illness by patients to cause severe destruction in tissues around teeth, and in most cases, it is an outcome of bacterial infiltration and an immune response in a host against bacteria and also the main cause of tooth loss.

Periodontitis is an inflammation occurring in the supportive tissue of teeth and is accompanied by gradual destruction of connective tissues as well as the loss of alveolar bone and periodontal ligaments. Depending on the rate of periodontal destruction, disease activity and host resistance, different types of bacteria are involved. Systemic diseases such as diabetes may aggravate periodontitis while host factors such as medication, smoking, pregnancy, obesity, and excessive stress are also risk factors. In addition, in the case of rapidly progressing periodontitis, it is known that genetic factors play a crucial role.

A method of measuring a probing pocket depth by inserting a probe into the sulcus, one of diagnostic methods currently used to diagnose periodontitis, is the most basic diagnostic method for periodontitis diagnosis, as a method of checking how much alveolar bone has been lost and determining a degree of inflammation in the gingiva. However, the method of measuring the probing pocket depth may cause errors depending on the tooth shape and a degree of gingival inflammation.

A method of radiographically determining the bone loss, along with probes for the probing pocket, is the most basic method of diagnosing periodontitis. However, it has the limitation in that it may show the alveolar bone loss in the mesiodistal surface of the tooth through a two-dimensional image without showing the alveolar bone loss in the buccolingual surface of the tooth where the image overlaps with the tooth. In addition, a method of identifying the probing pocket depth and bone loss on radiographs only shows the outcome of alveolar bone loss (loss of attachment) due to the progression of periodontitis before the time of diagnosis, without showing the active state of the current disease.

On the other hand, currently the most useful method that shows whether the gingiva has inflammation at the time of diagnosis is a method of inserting a probe into the sulcus between the tooth and the gingiva to check for bleeding. However, this method has the limitation in that there is a high probability of a false positive (despite the bleeding on probing, the actual gingiva may not have inflammation).

Such the conventional methods are cumbersome, time-consuming, and costly as the measurement is conducted by a specialist in the doctor's office, and some methods involve pain in the patient.

The periodontal disease, the main cause of tooth loss, is primarily caused by bacterial infection. Therefore, it is important to reduce the number of bacteria in the oral cavity and perform active treatment through early diagnosis. Thereby, there is a need to develop a method that is capable of easily diagnosing, for a periodontal disease with late detectable symptoms, whether the current condition of the gum is healthy without inflammation or has periodontitis.

In other words, if patients are able to determine the time of treatment early after checking the presence of a gum disease on their own, the time and cost for the treatment may be dramatically reduced.

### DISCLOSURE OF THE INVENTION

### Technical Goals

An object of the present disclosure is to provide a composition for diagnosing a periodontal disease.

Another object of the present disclosure is to provide a kit for diagnosing a periodontal disease.

Still another object of the present disclosure is to provide a method of providing information necessary for diagnosis of a periodontal disease.

### Technical Solutions

To achieve the above objects, the present disclosure provides a method of providing information necessary for diagnosis of a periodontal disease, including performing quantitative analysis by real-time polymerase chain reaction (real-time PCR) with one or more bacteria selected from the group consisting of Porphyromonas gingivalis, Tannerella forsythia, Prevotella intermedia, Porphyromonas endodontalis, and Filifactor alocis in a sample isolated from an individual; and comparing a bacterial % obtained through the quantitative analysis by real-time PCR with that selected from the group consisting of normal individuals and patients with gingivitis, moderate periodontitis, and severe periodontitis.

In addition, the present disclosure provides a method of providing information necessary for diagnosis of a periodontal disease, including performing quantitative analysis by real-time polymerase chain reaction (real-time PCR) with one or more bacteria selected from the group consisting of Porphyromonas gingivalis, Tannerella forsythia, Treponema denticola, Prevotella intermedia, Porphyromonas endodontalis, Filifactor alocis, Fusobacterium nucleatum, and Parvimonas micra in a sample isolated from an individual; and comparing a bacterial count obtained through the quantitative analysis by real-time PCR with that selected from the group consisting of normal individuals and patients with gingivitis, moderate periodontitis, and severe periodontitis.

In addition, the present disclosure provides a method of providing information necessary for diagnosis of a periodontal disease, including performing 16S rRNA sequencing analysis with one or more bacteria selected from the group consisting of Porphyromonas gingivalis, Tannerella forsythia, Treponema denticola, Prevotella intermedia, Porphyromonas endodontalis, Filifactor alocis, Fusobacterium nucleatum, and Parvimonas micra in a sample isolated from an individual; and comparing a bacterial % obtained through the 16S IRNA sequencing analysis with that selected from the group consisting of normal individuals and patients with gingivitis, moderate periodontitis, and severe periodontitis.

The present disclosure provides a composition for diagnosing a periodontal disease, including any one or more primer sets selected from the group consisting of a primer set represented by SEQ ID NOS: 1 and 2, a primer set represented by SEQ ID NOS: 3 and 4, a primer set represented by SEQ ID NOS: 5 and 6, a primer set represented by SEQ ID NOS:

7 and 8, a primer set represented by SEQ ID NOS: 9 and 10, a primer set represented by SEQ ID NOS: 11 and 12, a primer set represented by SEQ ID NOS: 13 and 14, and a primer set represented by SEQ ID NOS: 15 and 16.

In addition, the present disclosure provides a periodontal disease diagnostic kit including the composition.

### Advantageous Effects

The present disclosure seeks to distinguish the severity of a periodontal disease using colonies and a relative ratio of bacteria distributed in saliva. Since differences are shown in characteristics of bacterial colonies that vary by gingivitis, moderate periodontitis, and severe periodontitis, presented are the criteria to distinguish the severity of periodontal diseases using the same. Moreover, when collecting samples, saliva has the advantage of being safe, easy, and fast to access while minimizing discomfort in patients owing to non-invasiveness, and thus the development of diagnostic kits using saliva may help patients grasp a degree of progression of the periodontal disease on their own.

### BRIEF DESCRIPTION OF DRAWINGS

FIG. 1 shows a result of comparing the diversity of colonies in each group via 16s IRNA sequencing analysis.

FIG. 2 shows a result of comparing colonies at a phylum level in each group via 16s rRNA sequencing analysis.

FIG. 3 shows a result of comparing colonies at genera and species levels in each group via 16s rRNA sequencing analysis.

FIG. 4 shows results of comparing the % of specific bacteria in each group via quantitative analysis by real-time PCR.

FIG. 5 shows a result of comparing the count of specific bacteria in each group via quantitative analysis by real-time PCR.

FIG. 6 shows results of comparing a correlation between distribution of specific bacteria (16s rRNA sequencing %) and the sum of probing pocket depth in the whole oral cavity.

FIG. 7 shows results of comparing a correlation between distribution of specific bacteria (real-time PCR %) and the sum of probing pocket depth of the whole oral cavity.

FIG. 8 shows a result of comparing a correlation between distribution of specific bacteria (real-time PCR count) and the sum of probing pocket depth of the whole oral cavity.

FIG. 9 shows results of comparing distribution of specific bacteria by a correlation between 16s rRNA sequencing % and real-time PCR % (spearman's correlation coefficient).

### BEST MODE FOR CARRYING OUT THE INVENTION

Hereinafter, the present disclosure will be described in more detail.

The term “periodontal disease” as used herein may include diseases that occur in periodontal tissues, including gingival (gum), periodontal ligaments, and bone tissues around the teeth with diseases such as periodontitis and gingivitis. Depending on the severity of the disease, it is divided into gingivitis, moderate periodontitis, and severe periodontitis. It is known that, in the onset of the periodontal disease, the probing pocket is formed as the inflammation progresses to cause tissue damage, and the probing pocket becomes deeper with the severity of the periodontitis to cause inflammation in the periodontal ligament as the probing pocket deepens, finally resulting in induction of bone loss.

The term “diagnosis” as used herein refers to identification of the presence or characteristics of a pathological condition. For the purpose of the present disclosure, the diagnosis is to determine whether the periodontal disease has developed, a degree of disease progression, or whether there is a risk thereby.

The term “sample” as used herein refers to saliva secreted into the mouth from the salivary glands and, preferably, saliva that may be taken from a subject in a non-invasive manner.

The term “primer” as used herein refers to a short nucleic acid sequence having a short free 3′ hydroxyl group, which is capable of forming base pairs with a complementary template and acting as a starting point for template strand replication. The primer may initiate DNA synthesis in the presence of a reagent (i.e., DNA polymerase or reverse transcriptase) and four different nucleoside triphosphates for polymerization at an appropriate buffer and temperature. PCR conditions, as well as a length of sense and antisense primers may be appropriately selected according to the techniques known in the art.

In the present disclosure, the oligonucleotide used as a primer may also include a nucleotide analogue, e.g., phosphorothioate, alkyl phosphophothioate, or peptide nucleic acid, or may include an intercalating agent.

Accordingly, the present disclosure provides a method of providing information necessary for diagnosis of a periodontal disease, including performing quantitative analysis by real-time polymerase chain reaction (real-time PCR) with one or more bacteria selected from the group consisting of Porphyromonas gingivalis, Tannerella forsythia, Prevotella intermedia, Porphyromonas endodontalis, and Filifactor alocis in a sample isolated from an individual; and comparing a bacterial % obtained through the quantitative analysis by real-time PCR with that selected from the group consisting of normal individuals and patients with gingivitis, moderate periodontitis, and severe periodontitis.

In addition, the present disclosure provides a method of providing information necessary for diagnosis of a periodontal disease, including performing quantitative analysis by real-time polymerase chain reaction (real-time PCR) with one or more bacteria selected from the group consisting of Porphyromonas gingivalis, Tannerella forsythia, Treponema denticola, Prevotella intermedia, Porphyromonas endodontalis, Filifactor alocis, Fusobacterium nucleatum, and Parvimonas micra in a sample isolated from an individual; and comparing a bacterial count obtained through the quantitative analysis by real-time PCR with that selected from the group consisting of normal individuals and patients with gingivitis, moderate periodontitis, and severe periodontitis.

The comparing may include comparing the bacterial % or bacterial count obtained through the quantitative analysis by real-time PCR with a cut-off value set through the bacterial % and the bacterial count according to severity of the disease divided by a probing pocket, a degree of bleeding on probing, and a degree of alveolar bone resorption which indicate the severity of periodontitis.

The sample may be saliva.

The quantitative analysis by real-time polymerase chain reaction (real-time PCR) may measure an expression level of a target gene using any one or more primer sets selected from the group consisting of a primer set represented by SEQ ID NOS: 1 and 2, a primer set represented by SEQ ID NOS: 3 and 4, a primer set represented by SEQ ID NOS: 5 and 6, a primer set represented by SEQ ID NOS: 7 and 8, a primer set represented by SEQ ID NOS: 9 and 10, a primer set represented by SEQ ID NOS: 11 and 12, a primer set represented by SEQ ID NOS: 13 and 14, and a primer set represented by SEQ ID NOS: 15 and 16.

The primer set may be, but is not limited to, a primer set for real-time polymerase chain reaction (real-time PCR).

The target gene of the primer set represented by SEQ ID NOS: 1 and 2 may be rpoB in Porphyromonas gingivalis, the target gene of the primer set represented by SEQ ID NOS: 3 and 4 may be rpoB in Tannerella forsythia, the target gene of the primer set represented by SEQ ID NOS: 5 and 6 may be rpoB in Prevotella intermedia, the target gene of the primer set represented by SEQ ID NOS: 7 and 8 may be rpoB in Porphyromonas endodontalis, the target gene of the primer set represented by SEQ ID NOS: 9 and 10 may be 16s rRNA in Filifactor alocis, the target gene of the primer set represented by SEQ ID NOS: 11 and 12 may be rpoB in Treponema denticola, the target gene of the primer set represented by SEQ ID NOS: 13 and 14 may be rpoB in Fusobacterium nucleatum, and the target gene of the primer set represented by SEQ ID NOS: 15 and 16 may be fusA in Parvimonas micra.

As one example embodiment, the comparing may include distinguishing healthy gums and the periodontal disease if, based on a cut-off value of the bacterial % obtained through the quantitative analysis by real-time PCR, the cut-off value of the target gene of the primer set represented by SEQ ID NOS: 1 and 2 is greater than or equal to 0.0034, the cut-off value of the target gene of the primer set represented by SEQ ID NOS: 3 and 4 is greater than or equal to 0.0021, the cut-off value of the target gene of the primer set represented by SEQ ID NOS: 5 and 6 is greater than or equal to 0.0013, the cut-off value of the target gene of the primer set represented by SEQ ID NOS: 7 and 8 is greater than or equal to 0.0062, or the cut-off value of the target gene of the primer set represented by SEQ ID NOS: 9 and 10 is greater than or equal to 0.0004.

As another example embodiment, the comparing may include distinguishing healthy gum and the periodontal disease if, based on a cut-off value of the bacterial count obtained through the quantitative analysis by real-time PCR, the cut-off value of the target gene of the primer set represented by SEQ ID NOS: 1 and 2 is greater than or equal to 5, the cut-off value of the target gene of the primer set represented by SEQ ID NOS: 3 and 4 is greater than or equal to 10, the cut-off value of the target gene of the primer set represented by SEQ ID NOS: 5 and 6 is greater than or equal to 1, the cut-off value of the target gene of the primer set represented by SEQ ID NOS: 7 and 8 is greater than or equal to 9, the cut-off value of the target gene of the primer set represented by SEQ ID NOS: 9 and 10 is greater than or equal to 5, the cut-off value of the target gene of the primer set represented by SEQ ID NOS: 11 and 12 is greater than or equal to 11, or the cut-off value of the target gene of the primer set represented by SEQ ID NOS: 15 and 16 is greater than or equal to 94.

The cut-off value that may distinguish healthy gums and the periodontal disease is derived from a cut-off value of bacteria with an AUC value greater than or equal to 0.7 by obtaining an ROC curve and the AUC value to distinguish the healthy gums and gums with the periodontal disease based on the bacterial % or bacterial count from a saliva sample compared to the total bacteria of salivary bacteria collected from the oral cavity of the individual, and the periodontal disease refers to gingivitis, which is defined by a subject whose probing pocket depth of individual teeth is less than or equal to 3 mm and degree of bleeding on probing of an entire dentition is greater than or equal to 10%, as well as periodontitis which is accompanied by resorption of the alveolar bone.

As another example embodiment, the comparing may include distinguishing healthy gum/gingivitis and periodontitis if, based on a cut-off value of the bacterial % obtained through the quantitative analysis by real-time PCR, the cut-off value of the target gene of the primer set represented by SEQ ID NOS: 1 and 2 is greater than or equal to 0.0586, or the cut-off value of the target gene of the primer set represented by SEQ ID NOS: 3 and 4 is greater than or equal to 0.0048.

As another example embodiment, the comparing may include distinguishing the healthy gum/gingivitis and periodontitis if, based on a cut-off value of the bacterial count obtained through the quantitative analysis by real-time PCR, the cut-off value of the target gene of the primer set represented by SEQ ID NOS: 1 and 2 is greater than or equal to 33, the cut-off value of the target gene of the primer set represented by SEQ ID NOS: 3 and 4 is greater than or equal to 7, the cut-off value of the target gene of the primer set represented by SEQ ID NOS: 5 and 6 is greater than or equal to 3, the cut-off value of the target gene of the primer set represented by SEQ ID NOS: 7 and 8 is greater than or equal to 29, the cut-off value of the target gene of the primer set represented by SEQ ID NOS: 9 and 10 is greater than or equal to 16, the cut-off value of the target gene of the primer set represented by SEQ ID NOS: 11 and 12 is greater than or equal to 11, the cut-off value of the target gene of the primer set represented by SEQ ID NOS: 13 and 14 is greater than or equal to 529, or the cut-off value of the target gene of the primer set represented by SEQ ID NOS: 15 and 16 is greater than or equal to 233.

The cut-off value that may distinguish between healthy gum/gingivitis and periodontitis may be derived from a cut-off value of bacteria with an AUC value greater than or equal to 0.7 by obtaining an ROC curve and the AUC value to distinguish gums with periodontitis accompanied by resorption of alveolar bone from healthy gums and gums with gingivitis in a gum state not accompanied by resorption of alveolar bone based on the bacterial % or bacterial count from a saliva sample compared to the total bacteria of salivary bacteria collected from the oral cavity of the individual.

As another example embodiment, the comparing may include distinguishing healthy gum/gingivitis/moderate periodontitis and severe periodontitis if, based on the cut-off value of the bacterial % obtained through quantitative analysis by real-time polymerase chain reaction (real-time PCR), the cut-off value of the target gene of the primer set represented by SEQ ID NOS: 1 and 2 is greater than or equal to 0.1122, the cut-off value of the target gene of the primer set represented by SEQ ID NOS: 3 and 4 is greater than or equal to 0.0297, or the cut-off value of the target gene of the primer set represented by SEQ ID NOS: 5 and 6 is greater than or equal to 0.0037.

As another example embodiment, the comparing may include distinguishing healthy gum/gingivitis/moderate periodontitis and severe periodontitis if, based on the cut-off value of the bacterial count obtained through quantitative analysis by real-time polymerase chain reaction (real-time PCR), the cut-off value of the target gene of the primer set represented by SEQ ID NOS: 1 and 2 is greater than or equal to 838, the cut-off value of the target gene of the primer set represented by SEQ ID NOS: 3 and 4 is greater than or equal to 213, the cut-off value of the target gene of the primer set represented by SEQ ID NOS: 5 and 6 is greater than or equal to 18, the cut-off value of the target gene of the primer set represented by SEQ ID NOS: 9 and 10 is greater than or equal to 159, or the cut-off value of the target gene of the primer set represented by SEQ ID NOS: 13 and 14 is greater than or equal to 529.

The cut-off value that may distinguish the healthy gum/gingivitis/moderate periodontitis and severe periodontitis may be derived from a cut-off value of bacteria with an AUC value greater than or equal to 0.7 by obtaining an ROC curve and the AUC value to distinguish the healthy gums, gums with gingivitis and gums with moderate periodontitis from gums with severe periodontitis based on the bacterial % or bacterial count from a saliva sample compared to the total bacteria of salivary bacteria collected from the oral cavity of the individual, and the severe periodontitis may be periodontitis that shows probing pockets with a probing pocket depth greater than or equal to 6 mm locally and vertical bone resorption greater than or equal to 3 mm and has furcation-involved lesions caused by alveolar bone resorption in posterior teeth.

In addition, the present disclosure provides a method of providing information necessary for diagnosis of a periodontal disease, including performing 16S rRNA sequencing analysis with one or more bacteria selected from the group consisting of Porphyromonas gingivalis, Tannerella forsythia, Treponema denticola, Prevotella intermedia, Porphyromonas endodontalis, Filifactor alocis, Fusobacterium nucleatum, and Parvimonas micra in a sample isolated from an individual; and comparing a bacterial % obtained through the 16S IRNA sequencing analysis with that selected from the group consisting of normal individuals and patients with gingivitis, moderate periodontitis, and severe periodontitis.

As an example embodiment, cut-off values may be 0.5276 for Porphyromonas gingivalis, 0.0352 for Tannerella forsythia, 0.0044 for Treponema denticola, 0.0079 for Prevotella intermedia, 0.1662 for Porphyromonas endodontalis, 0.0141 for Filifactor alocis, 1.4741 for Fusobacterium nucleatum, 0.1722 for Parvimonas micra, or 0.6632 for Rothia dentocariosa, and, if it is greater than or equal to the corresponding cut-off value, healthy gums and periodontal diseases may be distinguished.

As another example embodiment, cut-off values may be 0.2258 for Porphyromonas gingivalis, 0.0611 for Tannerella forsythia, 0.1594 for Prevotella intermedia, 0.7348 for Porphyromonas endodontalis, 0.1141 for Filifactor alocis, 1.2996 for Fusobacterium nucleatum, 0.2103 for Parvimonas micra, or 0.3136 for Rothia dentocariosa, and, if the bacterial % is greater than or equal to the corresponding cut-off value, healthy gum/gingivitis and periodontitis may be distinguished.

As another example embodiment, cut-off values may be 1.4374 for Porphyromonas gingivalis, 0.1443 for Tannerella forsythia, 0.0294 for Prevotella intermedia, 0.7348 for Porphyromonas endodontalis, 0.1141 for Filifactor alocis, or 1.8720 for Fusobacterium nucleatum, and, if the bacterial % is greater than or equal to the corresponding cut-off value, healthy gum/gingivitis/moderate periodontitis and severe periodontitis may be distinguished.

The cut-off value is the same as described above.

The sample may be saliva.

The real-time polymerase chain reaction is a molecular biological polymerization method that amplifies a target using a target probe including a target primer and a label using cDNA as a template produced after reverse transcription of RNA into complementary DNA (cDNA) using reverse transcriptase, while quantitatively detecting a signal generated from the label of the target probe in the amplified target. This PCR method is well known in the art, and commercially available kits may also be used.

The PCR method may include analyzing a product amplified by PCR. The detection of the amplified product may be carried out by capillary electrophoresis, DNA chip, gel electrophoresis, radiometry, fluorimetry, or phosphorimetry. As one of the methods for detecting the amplified products, capillary electrophoresis may be performed. Capillary electrophoresis may be performed using, for example, an ABI sequencer. In addition, gel electrophoresis may be performed, in which agarose gel electrophoresis or acrylamide gel electrophoresis may be used depending on the size of the amplified product. In addition, in the fluorimetric method, PCR performed by labeling a 5′-end of the primer with Cy-5 or Cy-3 causes labeling with a fluorescent labelling material that detects the target sequence, and the fluorescence thus labeled may be measured using a fluorometer. In addition, in the radiometric method, radioisotopes such as ³²P or ³⁵S are added to a PCR reaction solution to label the amplified product during PCR, and then radioactivity may be measured using a radiometric instrument, e.g., a Geiger counter or a liquid scintillation counter.

The PCR method may include analyzing base sequences. Any of the methods known in the art may be used for the sequencing, specifically, but not limited to, automated sequencer may be used, or any one or more selected from among known methods may be used, such as pyrosequencing, restriction fragment length polymorphism (PCR-RELP), single strand conformation polymorphism (PCR-SSCP), specific sequence oligonucleotide (PCR-SSO), allele specific oligonucleotide (ASO) hybridization combined with a PCR-SSO method and dot hybridization, TaqMan-PCR, MALDI-TOF/MS, rolling circle amplification (RCA), high resolution melting (HRM), primer elongation, Southern blot hybridization, and dot hybridization.

In addition, the present disclosure provides a method of providing information necessary for diagnosis of a periodontal disease, including performing 16S rRNA sequencing analysis with one or more bacteria selected from the group consisting of Porphyromonas gingivalis, Tannerella forsythia, Treponema denticola, Prevotella intermedia, Porphyromonas endodontalis, Filifactor alocis, Fusobacterium nucleatum, Parvimonas micra, and Rothia dentocariosa in a sample isolated from an individual; and substituting a bacterial % obtained through the 16S rRNA sequencing analysis to Equations 1-1 to 1-4 below to calculate each value and determining gum disease severity corresponding to the Equation with the highest value thereamong as severity of the disease in a tooth:

$$Formula for healthy gums = - 3.52072 + ( - 0.026 \; 39 ) \times ( P . gingivalis \% ) + ( 0.839493 ) \times ( T . forsythia \% ) + ( 0.316224 ) \times ( T . denticola \% ) + ( - 0.986 \; 41 ) \times ( F . alocis \% ) + ( 0.646425 ) \times ( P . intermedia \% ) + ( 0.087085 ) \times ( P . endodontalis \% ) + ( - 0.087 \; 49 ) \times ( F . nucleatum \% ) + ( 1.391072 ) \times ( R . dentocariosa \% ) + ( 0.39933 ) \times ( P . micra \% ) \qquad [ Equation 1 - 1 ]$$

$$Formula for gums with gingivitis = - 1.9748 + ( - 0.011 \; 37 ) \times ( P . gingivalis \% ) + ( - 0.96119 ) \times ( T . forsythia \% ) + ( 0.0033 \; 46 ) \times ( T . denticola \% ) + ( - 0.25645 ) \times ( F . alocis \% ) + ( 0.568448 ) \times ( P . intermedia \% ) + ( 0.42965 ) \times ( P . endodontalis \% ) + ( 0.2009 \; 52 ) \times ( F . nucleatum \% ) + ( 0.519156 ) \times ( R . dentocariosa \% ) + ( - 0.52904 ) \times ( P . micra \% ) \qquad [ Equation 1 - 2 ]$$

$$Formula for gums with moderate periodontitis = - 1.9075 + ( 0.0070 \; 68 ) \times ( P . gingivalis \% ) + ( 1.141592 ) \times ( T . forsythia \% ) + ( - 0.2656 ) \times ( T . denticola \% ) + ( - 1.46872 ) \times ( F . alocis \% ) + ( 0.715288 ) \times ( P . intermedia \% ) + ( 0.77475 ) \times ( P . endodontalis \% ) + ( - 0.04183 ) \times ( F . nucleatum \% ) + ( 0.169773 ) \times ( R . dentocariosa \% ) + ( 1.520645 ) \times ( P . micra \% ) \qquad [ Equation 1 - 3 ]$$

$$Formula for gums with severe periodontitis = - 3.24579 + ( 0.296464 ) \times ( P . gingivalis \% ) + ( - 0.1594 ) \times ( T . forsythia \% ) + ( - 2.53253 ) \times ( T . denticola \% ) + ( - 0.11511 ) \times ( F . alocis \% ) + ( 1.83945 ) \times ( P . intermedia \% ) + ( 1.407898 ) \times ( P . endodontalis \% ) + ( 0.248094 ) \times ( F . nucleatum \% ) + ( 0.300277 ) \times ( R . dentocariosa \% ) + ( - 0.74747 ) \times ( P . micra \% ) \qquad [ Equation 1 - 4 ]$$

In addition, the present disclosure provides a method of providing information necessary for diagnosis of a periodontal disease, including performing quantitative analysis by real-time polymerase chain reaction (real-time PCR) with one or more bacteria selected from the group consisting of Porphyromonas gingivalis, Tannerella forsythia, Treponema denticola, Prevotella intermedia, Porphyromonas endodontalis, Filifactor alocis, Fusobacterium nucleatum, Parvimonas micra, and Rothia dentocariosa in a sample isolated from an individual; and substituting a bacterial % obtained through the quantitative analysis by real-time PCR to Equations 2-1 to 2-4 below to calculate each value and determining gum diseases severity corresponding to the Equation with the highest value thereamong as severity of the disease in a tooth:

$$Formula for healthy gums = - 3.60258 + ( - 0.051 ) \times ( P . gingivalis \% ) + ( 3.457539 ) \times ( T . forsythia \% ) + ( - 10.0746 ) \times ( T . denticola \% ) + ( - 1.25858 ) \times ( F . alocis \% ) + ( - 1.7208 ) \times ( P . intermedia \% ) + ( - 8.5888 ) \times ( P . endodontalis \% ) + ( 1.976449 ) \times ( F . nucleatum \% ) + ( 5.348558 ) \times ( R . dentocariosa \% ) + ( 0.063522 ) \times ( P . micra \% ) \qquad [ Equation 2 - 1 ]$$

$$Formula for gums with gingivitis = - 1.8156 + ( - 0.00877 ) \times ( P . gingivalis \% ) + ( 5.590504 ) \times ( T . forsythia \% ) + ( - 4.75712 ) \times ( T . denticola \% ) + ( - 1.4037 ) \times ( F . alocis \% ) + ( - 4.88342 ) \times ( P . intermedia \% ) + ( 5.253621 ) \times ( P . endodontalis \% ) + ( 0.331331 ) \times ( F . nucleatum \% ) + ( 3.02979 ) \times ( R . dentocariosa \% ) + ( 0.152853 ) \times ( P . micra \% ) \qquad [ Equation 2 - 2 ]$$

$$Formula for gums with moderate periodontitis = - 2.18027 + ( 0.14454 ) \times ( P . gingivalis \% ) + ( 12.54856 ) \times ( T . forsythia \% ) + ( 0.984939 ) \times ( T . denticola \% ) + ( - 4.98201 ) \times ( F . alocis \% ) + ( - 6.46175 ) \times ( P . intermedia \% ) + ( 3.469277 ) \times ( P . endodontalis \% ) + ( 1.076105 ) \times ( F . nucleatum \% ) + ( - 1.23915 ) \times ( R . dentocariosa \% ) + ( 0.468393 ) \times ( P . micra \% ) \qquad [ Equation 2 - 3 ]$$

$$Formula for gums with severe periodontitis = - 1.84498 + ( - 0.00562 ) \times ( P . gingivalis \% ) + ( 13.76487 ) \times ( T . forsythia \% ) + ( 2.553924 ) \times ( T . denticola \% ) + ( 0.054474 ) \times ( F . alocis \% ) + ( 7.646578 ) \times ( P . intermedia \% ) + ( 4.540174 ) \times ( P . endodontalis \% ) + ( - 0.48413 ) \times ( F . nucleatum \% ) + ( - 0.52195 ) \times ( R . dentocariosa \% ) + ( 0.21387 ) \times ( P . micra \% ) \qquad [ Equation 2 - 4 ]$$

In addition, the present disclosure provides a method of providing information necessary for diagnosis of a periodontal disease, including performing quantitative analysis by real-time polymerase chain reaction (real-time PCR) with one or more bacteria selected from the group consisting of Porphyromonas gingivalis, Tannerella forsythia, Treponema denticola, Prevotella intermedia, Porphyromonas endodontalis, Filifactor alocis, Fusobacterium nucleatum, Parvimonas micra, and Rothia dentocariosa in a sample isolated from an individual; and substituting a bacterial count obtained through the quantitative analysis by real-time PCR to Equations 3-1 to 3-4 below to calculate each value and determining gum disease severity corresponding to the Equation with the highest value thereamong as severity of the disease in a tooth:

$$Formula for healthy gums = - 2.30476 + ( - 1.49793 ) \times ( P . gingivalis count ) + ( 42.24108 ) \times ( T . forsythia count ) + ( - 51.0784 ) \times ( T . denticola count ) + ( 0.039624 ) \times ( F . alocis count ) + ( - 26.4862 ) \times ( P . intermedia count ) + ( - 90.5605 ) \times ( P . endodontalis count ) + ( 39.28479 ) \times ( F . nucleatum count ) + ( 17.65564 ) \times ( R . dentocariosa count ) + ( 4.278468 ) \times ( P . micra count ) \qquad [ Equation 3 - 1 ]$$

$$Formula for gums with gingivitis = - 1.62912 + ( - 0.76032 ) \times ( P . gingivalis count ) + ( 50.56172 ) \times ( T . forsythia count ) + ( - 65.645 ) \times ( T . denticola count ) + ( - 7.76311 ) \times ( F . alocis count ) + ( - 47.378 ) \times ( P . intermedia count ) + ( 45.18652 ) \times ( P . endodontalis count ) + ( 26.42666 ) \times ( F . nucleatum count ) + ( 47.9035 ) \times ( R . dentocariosa count ) + ( 5.394878 ) \times ( P . micra count ) \qquad [ Equation 3 - 2 ]$$

$$Formula for gums with moderate periodontitis = - 1.8899 + ( - 2.13304 ) \times ( P . gingivalis count ) + ( 121.1973 ) \times ( T . forsythia count ) + ( - 123.48 ) \times ( T . denticola count ) + ( - 11.1651 ) \times ( F . alocis count ) + ( - 83.2768 ) \times ( P . intermedia count ) + ( - 64.0856 ) \times ( P . endodontalis count ) + ( 43.52085 ) \times ( F . nucleatum count ) + ( 24.10447 ) \times ( R . dentocariosa count ) + ( 32.03642 ) \times ( P . micra count ) \qquad [ Equation 3 - 3 ]$$

$$Formula for gums with severe periodontitis = - 2.79975 + ( - 0.26374 ) \times ( P . gingivalis count ) + ( 366.5963 ) \times ( T . forsythia count ) + ( - 147.396 ) \times ( T . denticola count ) + ( 39.73937 ) \times ( F . alocis count ) + ( - 140.134 ) \times ( P . intermedia count ) + ( - 102.176 ) \times ( P . endodontalis count ) + ( 55.15521 ) \times ( F . nucleatum count ) + ( 123.9248 ) \times ( R . dentocariosa count ) + ( 29.36675 ) \times ( P . micra count ) \qquad [ Equation 3 - 4 ]$$

In addition, the present disclosure provides a composition for diagnosing a periodontal disease, including any one or more primer sets selected from the group consisting of a primer set represented by SEQ ID NOS: 1 and 2, a primer set represented by SEQ ID NOS: 3 and 4, a primer set represented by SEQ ID NOS: 5 and 6, a primer set represented by SEQ ID NOS: 7 and 8, a primer set represented by SEQ ID NOS: 9 and 10, a primer set represented by SEQ ID NOS: 11 and 12, a primer set represented by SEQ ID NOS: 13 and 14, and a primer set represented by SEQ ID NOS: 15 and 16.

In addition, the present disclosure provides a periodontal disease diagnostic kit including the composition for diagnosing a periodontal disease.

The kit of the present disclosure may include, in addition to the primer set described above, conventional components included in a PCR kit. Conventional components included in the kit may be reaction buffers, polymerases, dNTPs (dATP, dCTP, dGTP, and dTTP), and cofactors such as Mg²⁺. A variety of DNA polymerases may be used in amplification steps, including the Klenow fragment of E. coli DNA polymerase I, thermostable DNA polymerase, and bacteriophage T7 DNA polymerase. Preferably, polymerase is a thermostable DNA polymerase that is obtainable from a variety of bacterial species. Most of the polymerases may be isolated from the bacteria themselves or commercially available.

Modes for Carrying Out the Invention

Hereinafter, the present disclosure will be described in detail through example embodiments. These example embodiments are merely for illustrating the present disclosure more specifically, and it will be apparent to those skilled in the art that the scope of the present disclosure is not limited by these example embodiments according to the gist of the present disclosure.

### EXAMPLE 1

### Selection of Study Subjects

Patients who came to the periodontal department of Ajou University Dental Hospital for examination, scaling, and periodontal treatment were recruited as study subjects.

This study was conducted after receiving informed consent from all study subjects. Those who had orthodontic appliances in the oral cavity, other systemic medical history that may affect the progression of periodontitis, and a smoking habit, as well as those who had taken antibiotics and antimicrobial anti-inflammatory drugs in the three months prior to saliva collection were excluded from the study subjects.

Periodontitis was diagnosed through probing pocket measurement performed before periodontal treatment. The classification of healthiness and severity of a disease in patients was based on the results of a conference jointly held by the European Society of Periodontology and the American Society of Periodontology in 2017 and the new classification criteria (J Periodontol. 2018 June;89 Suppl 1:S159-S172.).

The definitions of healthy gums, gums with gingivitis, gums with moderate periodontitis, and gums with severe periodontitis are as follows.
\textcircled{1} Healthy gums: Subjects who have a probing pocket depth less than or equal to 3 mm and a bleeding on probing (BOP) index less than 10%
\textcircled{2} Gums with gingivitis: Subjects who have a probing pocket depth less than or equal to 3 mm and a BOP index greater than or equal to 10% on probing
\textcircled{3} Gums with moderate periodontitis: Subjects who have a probing pocket depth of 3-4 mm and a maximum probing pocket depth less than or equal to 5 mm and show mostly horizontal bone resorption without experiencing tooth extraction due to periodontitis (alveolar bone resorption is coronal third (15˜33%))
\textcircled{4} Gums with severe periodontitis: Subjects who have a probing pocket depth greater than or equal to 6 mm locally and show vertical bone resorption greater than or equal to 3 mm with posterior furcation-involved lesions (Class II or III)

### EXAMPLE 2

### Evaluation of Clinical Indicators

### 1) Clinical Indicators

The clinical indicators for periodontitis of the study subjects were evaluated by

the plaque index for the whole oral cavity, gingival index, probing pocket depth, clinical attachment level, modified sulcus bleeding index, and degree of bleeding on probing.

### \textcircled{1} Plaque Index (PI): A Degree of Bacterial Film Deposition on a Surface of the Tooth

Score 0=No plaque
Score 1=Plaque that is scratched by a probe
Score 2=Obvious plaque visible to the eye
Score 3=Abundant plaque
   (2) Gingival Index (GI): A degree of inflammation in the gingiva
Score 0=No inflammation
Score 1=Mild inflammation accompanied by changes in the color and appearance in the gingiva, but no bleeding on probing
Score 2=Moderate inflammation accompanied by changes in the color and appearance in the gingiva with bleeding on probing
Score 3=Severe inflammation with natural abundant bleeding

### \textcircled{3} Probing Pocket Depth (PD)

It is a value obtained by measuring, using a periodontal probe (Hu-Friedy PCP 10, USA), a length from the gingival margin to the base of the probing pocket for a total of 6 parts (buccal mesial surface, buccal center, buccal distal surface, lingual mesial surface, lingual center, lingual distal surface), 3 parts each from the outside and inside of one tooth, meaning that the higher the value, the more severe the edema, alveolar bone destruction, and periodontitis.

### \textcircled{4} Clinical Attachment Level (CAL)

It is a value obtained by measuring, using a periodontal probe, a length from the

cementoenamel junction to the base of the probing pocket for a total of 6 parts, 3 parts each from the outside and inside of one tooth and also a value obtained by adding the probing pocket depth with the amount of periodontal recession if there is a gingival recession, meaning that the higher the value, the more severe the periodontitis, just like the meaning of probing pocket depth.

\textcircled{5} Modified Sulcus Bleeding Index (mSBI)

On the basis of conventional methods (Mombelli et al. 1987), it is a value to evaluate the bleeding index within the modified sulcus using a periodontal probe for a total of 6 parts, 3 parts each from the outside and inside of one tooth, meaning that the higher the value, the more severe the periodontitis.
Score 0=A case with no bleeding
Score 1=A case with petechial hemorrhage
Score 2=A case in which bleeding is widespread along the gingival margin
Score 3=A case with abundant bleeding that oozes out

### \textcircled{6} Bleeding On Probing (BOP)

It is a value that shows an average of all teeth and is derived by recording the bleeding using a periodontal probe for a total of 6 parts, 3 parts each from the outside and inside of one tooth and then obtaining the percentage of spots where the bleeding is detected, meaning that the higher the value, the more severe the inflammation in the gingiva of the mouth.

### 2) Evaluation of Clinical Indicators

As shown in Table 1 below, the study subjects were divided into four groups and evaluated for clinical indicators. Performance was conducted on 8 patients with healthy gums, 16 in the gingivitis group, 19 in the moderate periodontitis group, and 29 in the severe periodontitis group, and significant differences were found in the clinical indicators presented by each group.

TABLE 1

| | | | Moderate | Sever | | |
|---|---|---|---|---|---|---|
| | Healthy | Gingivitis | periodontitis | periodontitis | | |
| | (n = 8) | (n = 16) | (n = 19) | (n = 29) | p | pReg |
| Sum of PI | [symbol] ± [symbol] | 34.94 ± 23.52 | 61.11 ± 23.85 | 61.86 ± [symbol] | <.001 | <.001 |
| Sum of PD | [symbol] ± 28.76 | 363.94 ± 38.14 | 430.68 ± 40.19 | 531.97 ± 95.58 | <.001 | <.001 |
| Mean PD | 1.92 ± 0.14 | 2.29 ± 0.25 | 2.63 ± 0.20 | 3.39 ± 0.63 | <.001 | <.001 |
| Sum of mSBI | 15.50 ± 0.53 | 106.75 ± 56.25 | 147.32 ± [symbol] | 178.10 ± [symbol] | <.001 | <.001 |
| Sum of GI | 79.63 ± 4.03 | 96.50 ± 13.80 | 109.26 ± 10.77 | 112.48 ± [symbol] | <.001 | <.001 |
| Mean GI | 1.46 ± [symbol] | 1.82 ± 0.25 | [symbol] ± 0.18 | 2.14 ± 0.27 | <.001 | <.001 |
| Mean CAL | 2.23 ± 0.26 | 2.37 ± 0.23 | 2.76 ± 0.18 | 3.98 ± 0.85 | <.001 | <.001 |
| BOP(%) | 9.38 ± 0.32 | 51.57 ± 19.88 | 64.62 ± 11.88 | 71.77 ± 16.42 | <.001 | <.001 |

PI, plaque index;
PD, [symbol] depth;
mSBI, modified [symbol] bleeding index;
GI, gingival index;
CAL, clinical [symbol] level;
BOP, bleeding on probing [symbol]
indicates data missing or illegible when filed

### EXAMPLE 3

### Collection of Saliva

All study subjects were advised to refrain from eating, brushing, and rinsing their mouths (mouthwash) for one hour before saliva collection. Saliva was collected in a non-irritable saliva collection mode by gargling with 10 ml of normal saline for 1 minute and then spitting it out.

### EXAMPLE 4

### 16S rRNA Sequencing (Metataxonomics) Analysis

Out of a total of 10 ml of saliva collected, 8 ml was taken and centrifuged. DNA extraction and sequencing analysis (16S rRNA sequencing) of the entire sediment (including bacteria) were performed by ChunLab, Inc. After DNA extraction, the V3 and V4 regions were amplified using primers targeting the V3 and V4 regions of the bacterial 16S rRNA, and the PCR products were analyzed by the Illumina MiSeq sequencing system.

Using EzBioCloud (https://www.ezbiocloud.net/apps), a web-based analysis platform provided by ChunLab, the taxonomic profiles of bacterial colonies were analyzed and compared in the healthy, gingivitis, moderate periodontitis, and severe periodontitis groups. The Wilcoxon rank-sum test was used to compare the number of Operational Taxonomic Units (OTUs), Chaol index, Shannon index, and phylogenetic diversity for each group. In addition, the Kruskal-Wallis H test was performed to assess the difference in the predominant OTU in each group. OTU is a taxonomic unit that groups similar sequences by species from DNA sequencing results, and the Chaol and Shannon indices are indicators that assess the abundance and uniformity of colony, respectively.

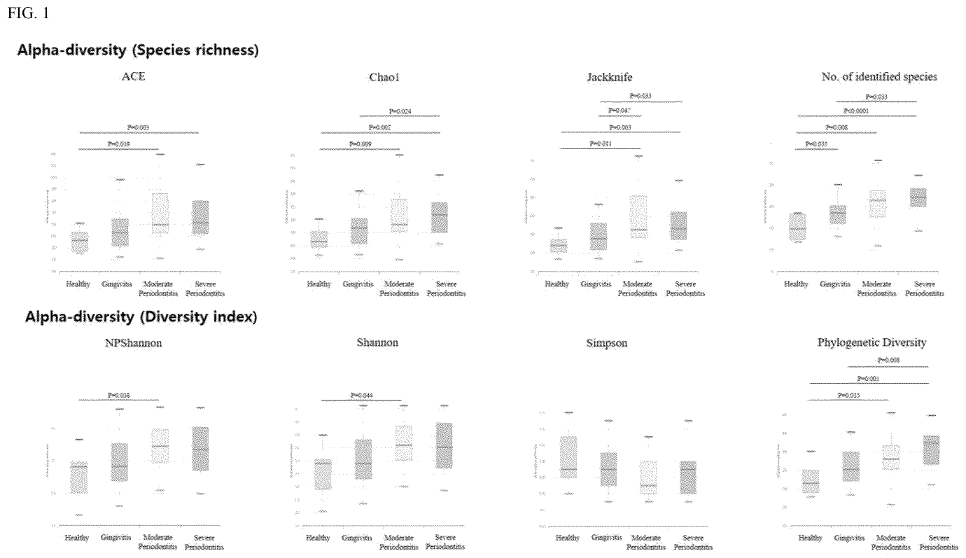

FIG. 1 shows a result of comparing a difference in the diversity in each group via 16s rRNA sequencing analysis. When it comes to diversity analysis, there are three main types of analysis (alpha diversity, beta diversity, and gamma diversity), of which alpha diversity is one that analyzes the distribution of various microorganisms present in a sample, referring to the average species diversity in a local-scale place or habitat. ACE, Chaol, Jackknife, No. of identified species, NPShannon, Shannon, Simpson, and phylogenetic diversity presented are all examples showing alpha diversity. As shown in the results, it was found that the diversity increases with the severity of the disease, and various types of bacteria inhabit in the disease group than the healthy group.

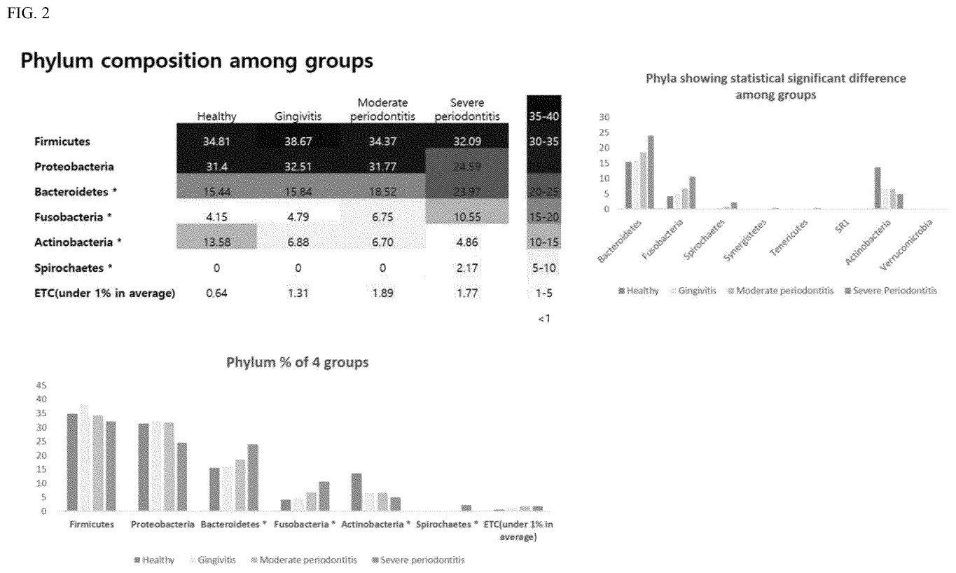

FIG. 2 shows a result of a comparing colonies at a phylum level in each group via 16s rRNA sequencing analysis. Bacteroidetes, Fusobacteria, and Spirochaetes were found to increase significantly with the severity of the disease, while Actinobacteria was present in high concentrations in healthy gums and decreased significantly as the severity of the disease increased. In addition, high concentrations of Firmicutes and Proteobacteria were present in saliva, with a tendency to decrease as the severity of the disease increased.

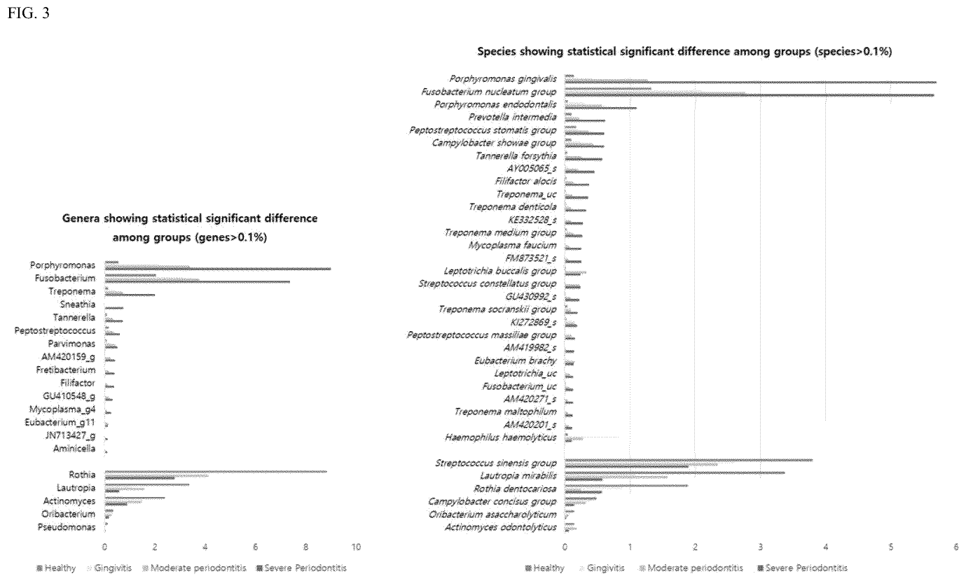

FIG. 3 shows a result of a comparing colonies at genera and species levels in each group via 16s rRNA sequencing analysis. 15 genera including Porphyromonas, Fusobacterium, and Treponema were present in high proportions in the disease group, and 5 genera, including Rothia, Lautropia, and Actinomyces, were present in high proportions in the healthy group.

In addition, 29 species, including Porphyromonas gingivalis, were present in high proportion in the diseased group, and 6 species, including Streptococcus sinensis group, were present in high proportion in the healthy group. At the species level, it is indicated that bacterial species labeled as a group at the end of their names may include similar 16S IRNA bacterial species other than that species. The 16S rRNA sequencing technique reads only a part of the total sequence and determines the similarity to the base sequence in the published database by 97%, so it is sometimes determined to be identical to a bacterium other than a specific bacterium, in which case the most representative bacterial species are written with a group at the end of the name. For example, the F. nucleatum group may include all the following bacteria: F. nucleatum subspecies nucleatum, vincentii, polymorphum, fusiforme, animalis, F. simiae, F. canifelinum, F. hwasookii.

### EXAMPLE 4

### Quantitative Analysis of Specific Bacteria Using Real-Time (RT) PCR

Based on the results of 16S rRNA sequencing, the following 9 types of bacteria were selected that showed statistical significance for each group, and quantitative analysis was performed by real-time PCR using a species-specific primer:
Porphyromonas gingivalis, Tannerella forsythia, Treponema denticola, Prevotella intermedia, Porphyromonas endodontalis, Filifactor alocis, Fusobacterium nucleatum, Parvimonas micra, Rothia dentocariosa

200 μL of saliva was taken to be centrifuged, and DNA extraction of the entire sediment was performed by ChunLab Inc. The total bacterial count was analyzed via real-time PCR using a universal primer (FEMS Immunol Med Microbiol. 2003 Oct. 24;39(1):81-6.) targeting 16S rRNA. Considering the copy number of 16s rRNA in a single bacterium, the result value of real-time PCR was divided by 6 and considered as a single bacterium.

Of the 9 species of bacteria, the bacterial counts were calculated on the basis of formulas described in the paper (J Microbiol. 2011 April; 49(2):315-9.) for the bacterium P. gingivalis, the paper (International Journal of Oral Biology Vol.40 No.4 pp.205-210) for bacterium P. intermedia, and the paper (Arch Microbiol. 2013 July; 195(7):473-82.) for 5 species of bacteria (T. forsythia, T. denticola, P. endodontalis, F. nuclatum, R. dentocariosa). For the other two species of bacteria (F. alocis, P. micra), in reference to the paper (Sci Rep. 2016 Sep. 19;6:33638., Front Microbiol. 2017 Aug. 9;8:1443.), the DNA of the actual bacteria was extracted, the standard curve was set by performing real-time PCR for each concentration, and thereby the real-time PCR of a saliva sample to be identified was performed by obtaining the Equation (first-order equation) for the real-time PCR value related to the bacterial concentration, followed by quantitative analysis on the bacteria in the sample based on this Equation. The Equations were obtained as the average value of the same experiment repeated three times independently.

Each real-time PCR was performed in a total volume of 20 μL, including 2 μL of genomic DNA, 2 μL of forward and reverse primers, 6 μL of sterile DNase-RNase-free water, and 10 μL of TOP real™ qPCR 2X PreMIX. All data were analyzed using TOP real™ qPCR 2X PreMIX (SYBR Green) kit (Enzynomics, Daejeon, Korea) and Exicycler™ 96 V4 Real-Time Quantitative Thermal Block (Bioneer, Daejeon, Korea). The sequences of the primer used in the experiment are shown in Table 2 below.

TABLE 2

| | Primer | | Sequence |
|---|---|---|---|
| Bacteria | Target | Sequence (5′ to 3′) | Number |
| P. gingivalis | rpoB | Pg-F: GGA AGA GAA GAC CGT AGC ACA | 1 |
| | | AGG A | |
| | | Pg-R: GAG TAG GCG AAA CGT CCA TCA | 2 |
| | | GGT C | |
| T. forsythia | rpoB | Tf-F2: GGATTGACCACCGGCGAAGACA | 3 |
| | | Tf-R2: | 4 |
| | | CGGACACGACGGTTACTCAAATGG | |
| P. intermedia | rpoB | RTPi-F: ACC CAT TGG CAG AAG TTA CG | 5 |
| | | RTPi-R: TCA ATA GGA CAC AAG CGA | 6 |
| | | CCA TAG | |
| P. endodontalis | rpoB | Pe-F1: | 7 |
| | | AGCAGAGTTCCGTCGTCGTATTCA | |
| | | Pe-R1: GCGCCCTGCCATCTTGTCTC | 8 |
| F. alocis | 16S rRNA | F: AACCGGAGCAAAACTGAGAA | 9 |
| | | R: CCGTCCGCCACTAACTTCTA | 10 |
| T. denticola | rpoB | Td-F2: | 11 |
| | | CGGGCGTGCATCTTGTCGTCTAC | |
| | | Td-R2: CTTAACCGGCCGCCTCTTTGAA | 12 |
| F. nucleatum | rpoB | Fn-F1: | 13 |
| | | ACCTAAGGGAGAAACAGAACCA | |
| | | Fn-R1: CCTGCCTTTAATTCATCTCCAT | 14 |
| P. micra | fusA | F: AGAATCAATTTCTCAAGGTGCAG | 15 |
| | (single copy) | R: CAATGTCCATATTGTCCACTACCA | 16 |
| R. dentocariosa | rpoB | Rd-F8: CTGGGCAAAGCGTCTGGAAAAC | 17 |
| | | Rd-R8: | 18 |
| | | GAAATCACGAATCGGGGAAATCTC | |
| Universal primer | 16S rRNA | F: GTG STG CAY GGY TGT CGT CA | 19 |
| | | R: ACG TCR TCC MCA CCT TCC TC | 20 |

### EXAMPLE 5

### Statistical Analysis

The Kruskal-Wallis H test was performed to analyze whether the results of real-time PCR for each of the 9 bacteria showed differences in each group. Spearman's correlation coefficient analysis was performed to determine the correlation between 16S rRNA sequencing (%) and real-time PCR (%) in specific bacteria.

Probing pockets were measured for the entire tooth by 6 parts per tooth, and the sum thereof was evaluated by simple linear regression analysis for the correlation between the sum of the probing pocket depth and the concentration of specific bacteria (16S rRNA sequencing %, real-time PCR %, real-time PCR count) as a basis that may reflect the severity of periodontitis in the entire dentition (oral cavity) (%: a ratio of the bacteria to the total bacteria, count: the number of bacteria in 15 ul out of the total saliva (10 ml) collected).

To obtain the optimal cut-off value for each bacterium that distinguishes the severity of the disease, Receiver Operating Characteristic (ROC) curves were drawn. The cut-off value for each bacterium that distinguishes the degree of diseases by each periodontitis severity was obtained, the cut-off value was presented for the value with the highest Area under the ROC Curve (AUC), and the sensitivity and specificity were presented.

### 1) Quantitative Analysis Results of Specific Bacteria By Real-Time PCR

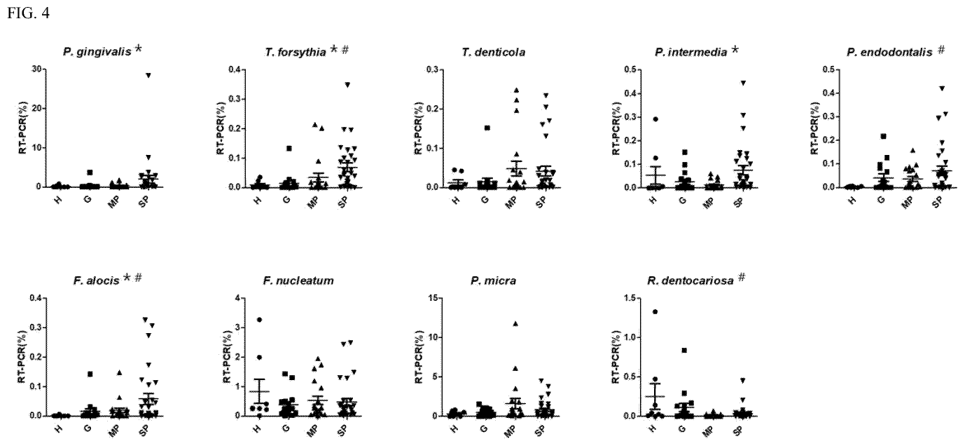

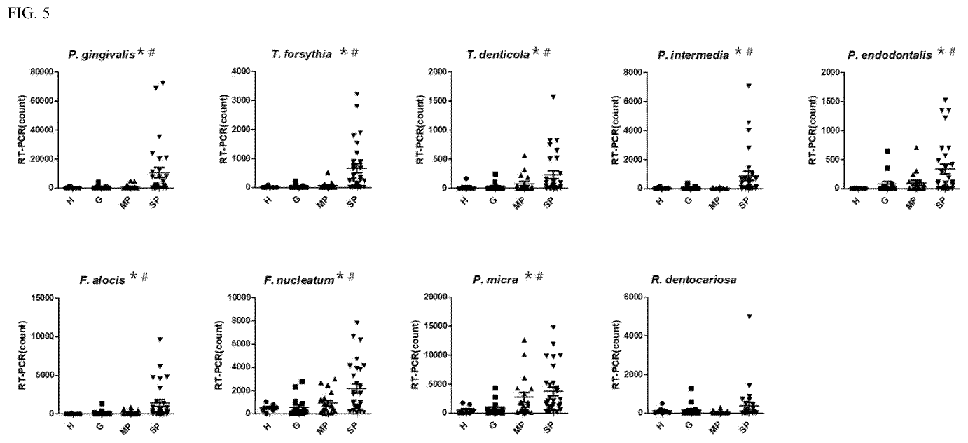

FIGS. 4 and 5 show results of comparing the % and count of specific bacteria in each group based on the quantitative analysis by real-time PCR of specific bacteria. The graph values in FIGS. 4 and 5 are shown in Table 3 and Table 4 below, respectively. In regard to the %, it was found that P. gingivalis, T. forsythia, F. alocis, P. intermedia, and P. endodontalis were present in high concentrations in severe periodontitis, and R. dentocariosa was present in high concentrations in the healthy group. In regard to the count results, it was found that the remaining bacteria, except for R. dentocariosa, were present in significantly higher concentrations in saliva as the severity of the disease increased.

For reference, pReg represents p-value of whether the mean increases or decreases, given with values of 0, 1, 2, and 3 from the healthy group to the severe periodontitis group, respectively. In other words, significance in p-value means that there is a difference between groups, and significance in pReg means that there is a tendency for the value to increase or decrease as it progresses to the healthy, gingivitis, moderate periodontitis, and severe periodontitis groups.

TABLE 3

| | | | | Moderate | Severe | | |
|---|---|---|---|---|---|---|---|
| var | Total | Healthy | Gingivitis | periodontitis | periodontitis | p | pReg |
| [symbol] | 0.9750 ± [symbol] | 0.1250 ± 0.2790 | 0.2870 ± 0.9190 | [symbol] ± 0.5500 | [symbol] ± 5.3300 | [symbol] | 0.075 |
| [symbol] | 0.0410 ± [symbol] | [symbol] ± [symbol] | 0.0140 ± 0.0330 | 0.0340 ± [symbol] | 0.0690 ± 0.0790 | [symbol] | 0.002 |
| [symbol] | 0.0340 ± 0.0630 | 0.0120 ± 0.0200 | 0.0140 ± 0.0370 | [symbol] ± 0.0810 | 0.0420 ± 0.0670 | 0.087 | 0.113 |
| [symbol] | 0.0320 ± 0.0680 | 0.0010 ± 0.0020 | [symbol] ± 0.0350 | 0.0190 ± [symbol] | [symbol] ± 0.0950 | 0.003 | 0.010 |
| P. intermedia | 0.0450 ± 0.0820 | [symbol] ± 0.1060 | 0.0250 ± 0.0440 | 0.0130 ± 0.0190 | 0.0750 ± 0.1050 | [symbol] | 0.144 |
| [symbol] | 0.0480 ± 0.0800 | [symbol] ± 0.0020 | 0.0420 ± 0.0620 | 0.0370 ± 0.0450 | 0.0710 ± 0.1080 | [symbol] | [symbol] |
| F. nucleatum | 0.5070 ± [symbol] | [symbol] ± 1.1680 | 0.3750 ± 0.4240 | 0.5200 ± [symbol] | 0.4750 ± 0.6719 | 0.805 | 0.506 |
| [symbol] | [symbol] ± 0.1980 | 0.2550 ± [symbol] | 0.1130 ± 0.2100 | 0.0150 ± 0.0170 | 0.0370 ± [symbol] | 0.127 | 0.007 |
| P. micra | 0.9210 ± [symbol] | [symbol] ± [symbol] | 0.4970 ± 0.4640 | 1.6360 ± 2.8910 | [symbol] ± [symbol] | [symbol] | [symbol] |

[symbol]

indicates data missing or illegible when filed

TABLE 4

| | | | | Moderate | Severe | | |
|---|---|---|---|---|---|---|---|
| var | Total | Healthy | Gingivitis | periodontitis | periodontitis | p | pReg |
| [symbol] | [symbol] ± 12832.25 | 225.94 ± [symbol] | [symbol] ± 974.23 | [symbol] ± 1534.49 | 10557.34 ± [symbol] | <.001 | 0.005 |
| [symbol] | 287.45 ± [symbol] | [symbol] ± 31.65 | 22.99 ± 53.13 | [symbol] ± 118.56 | 658.11 ± [symbol] | <.001 | <.001 |
| [symbol] | 126.21 ± 265.14 | 24.89 ± 59.68 | 25.56 ± 64.76 | 83.25 ± 148.13 | 237.84 ± [symbol] | 0.001 | 0.005 |
| [symbol] | 668.44 ± 1616.03 | 18.31 ± 48.79 | 156.46 ± 340.65 | 219.00 ± [symbol] | 1424.70 ± 2346.83 | <.001 | [symbol] |
| P. intermedia | 374.76 ± [symbol] | 31.84 ± 56.33 | [symbol] ± 120.30 | 20.56 ± 30.08 | 877.29 ± [symbol] | <.001 | 0.019 |
| [symbol] | 178.75 ± 343.53 | [symbol] ± 2.91 | 74.99 ± 174.86 | 99.83 ± 170.79 | 336.21 ± 468.20 | [symbol] | [symbol] |
| F. nucleatum | [symbol] ± 1671.55 | 494.77 ± 314.96 | 560.48 ± 828.13 | 894.51 ± 938.53 | 2162.64 ± 2178.74 | 0.003 | <.001 |
| [symbol] | [symbol] ± 633.18 | 119.72 ± 165.92 | 155.49 ± 331.57 | 55.57 ± 72.64 | [symbol] ± 949.17 | [symbol] | 0.213 |
| P. micra | 2246.17 ± [symbol] | 486.64 ± 727.69 | 744.07 ± 1196.85 | 2706.94 ± 3504.89 | 3754.96 ± 4010.82 | <.001 | 0.001 |

[symbol]

indicates data missing or illegible when filed

### 2) Results of Analyzing the Correlation Between Distribution of Specific Bacteria and the Sum of the Probing Pocket Depth

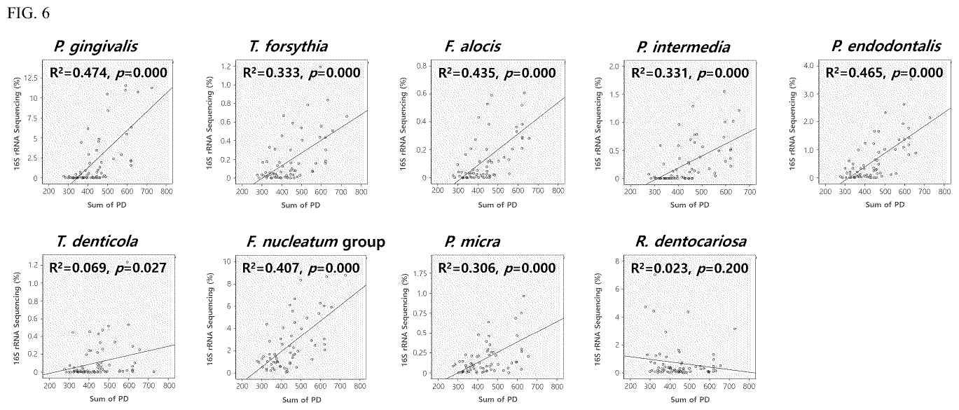

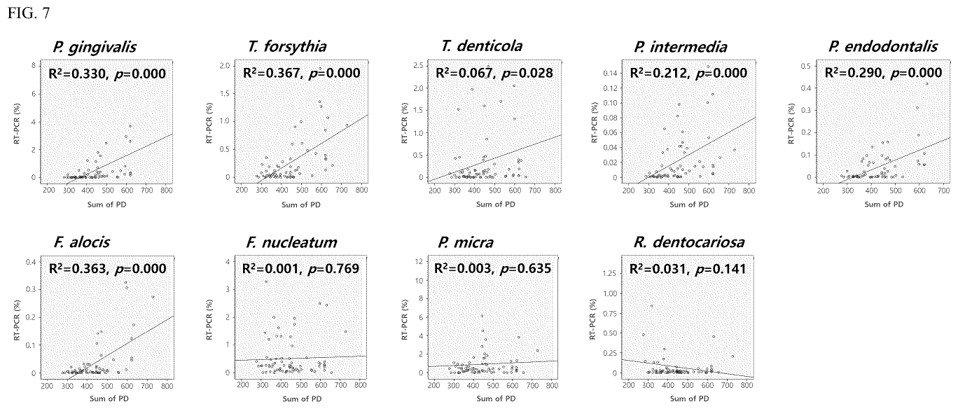

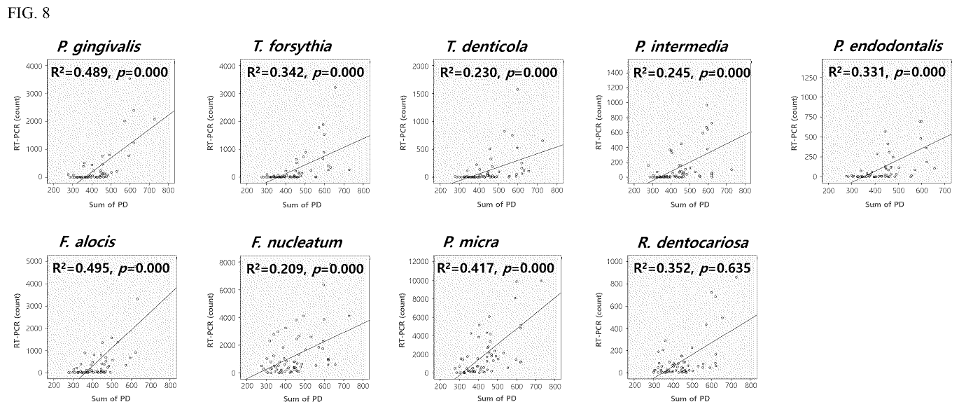

FIGS. 6 to 8 show analysis on the correlation between distribution of specific bacteria (16S rRNA sequencing (%) and real-time PCR (% or count) and the sum of the probing pocket depth in the whole oral cavity, and the values of each graph are shown in Table 5 below.

In analyzing the correlation with the distribution of specific bacteria, the sum of the probing pocket depth among several clinical indicators (PD, mSBI, BOP %, CAL, PI, GI, etc.) that distinguish the severity of diseases was selected because the deeper the probing pocket depth, the more dysbiosis of the subgingival flora was advanced, assuming that the result of the colonization collapse of these subgingival flora would be reflected in the saliva. The table presented shows the correlation between the bacterial concentration and the sum of the probing pocket depth in the whole oral cavity, meaning that the higher the R2 value, the higher the correlation.

In the case of 16S rRNA sequencing (%), a negative correlation with R. dentocariosa was found to have no statistical significance, while a significant positive correlation was found in all remaining bacteria. In other words, it may be said that the deeper the overall probing pocket, the higher the concentration of the corresponding bacteria.

In the case of real-time PCR (%), F. nucleatum, P. micra, and R. dentocariosa did not show statistically significant results, but a significant positive correlation was shown in the remaining bacteria.

In the case of real-time PCR (count), there was a positive correlation over all bacteria.

TABLE 5

| | | [symbol] | [symbol] | [symbol] | [symbol] | P. intermedia | [symbol] | F. nucleatum | P. micra | [symbol] |
|---|---|---|---|---|---|---|---|---|---|---|
| NGS | R2 | .474 | .333 | .069 | .435 | .331 | .465 | .407 | .306 | .023 |
| (%) | beta | .023 | .001 | .001 | .001 | .002 | .005 | .014 | .001 | [symbol] |
| | P-value | .000 | .000 | .027 | [symbol] | .000 | .000 | .000 | .000 | .200 |
| RT-PCR | R2 | .330 | .367 | .067 | .363 | .212 | .290 | .001 | [symbol] | .031 |
| (%) | beta | .007 | .000 | .000 | .000 | .000 | .000 | .000 | .001 | .000 |
| | P-value | .000 | .000 | [symbol] | .000 | [symbol] | .000 | [symbol] | .635 | .141 |
| RT-PCR | R2 | .489 | .342 | .230 | .495 | .245 | .331 | .209 | .417 | .352 |
| (count) | beta | 50.814 | 3.146 | 1.203 | 8.136 | 1.233 | 1.415 | 6.671 | 16.781 | 1.072 |
| | P-value | .000 | .000 | .000 | .000 | .000 | [symbol] | .000 | .000 | [symbol] |

[symbol]

indicates data missing or illegible when filed

3) Results of Analyzing on the Correlation with 16S rRNA Sequencing (%) and Real-Time PCR (%) of Specific Bacteria

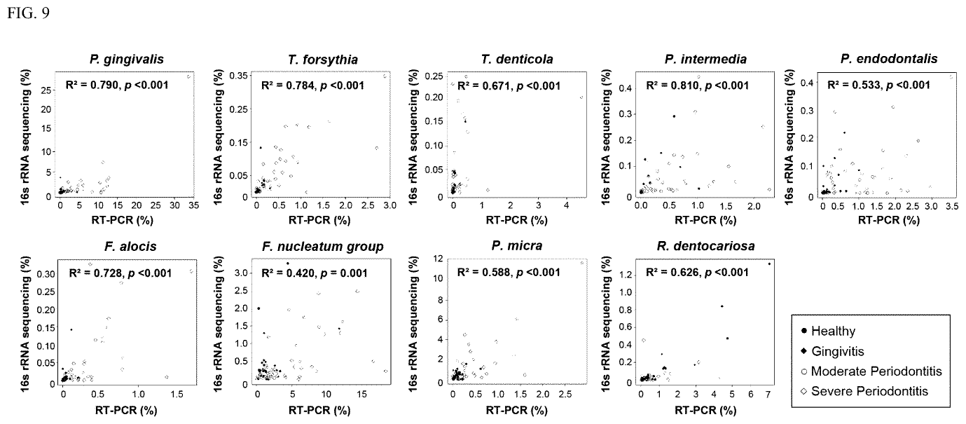

In the case of the 16S rRNA sequencing technique, if it reads only a part of the entire base sequence and shows a 97% similarity to the base sequence in the published database, it is determined to be the bacterium, such that it may be relatively lower in accuracy than real-time PCR. Thereby, the result of analyzing the correlation between the % value of the bacteria compared to the total bacteria obtained from the results of 16S IRNA sequencing (Next Generation Sequencing, NGS) and the % of the bacteria compared to the total bacteria obtained from the real-time PCR results is shown in FIG. 9.

As a result of analysis, it appeared that all had a statistically significant correlation, especially P. gingivalis and P. intermedia had high significance while F. nucleatum had relatively low significance. The results of 16S rRNA sequencing for F. nucleatum may be seen as a group to include all F. nucleatum subspecies nucleatum, vincentii, polymorphum, fusiforme, animalis, F. Simiae, F. canifelinum, and F. hwasookii. In the case of the species-specific primer of F. nucleatum used in real-time PCR also, the correlation between results from the two experimental methods may be low because it is not the primer for F. nucleatum alone, but rather a primer that detects both F. nucleatum and F. simiae.

### 4) Establishment of Reference Values for Each Bacterium to Distinguish the Severity of Diseases and Diagnosis Methods

### (1) (Healthy Gums) vs. (Gingivitis, Moderate Periodontitis, Severe Periodontitis)

It is a method of distinguishing healthy gums and periodontal diseases (gingivitis and periodontitis). The periodontal disease is broadly divided into gingivitis and periodontitis, with gingivitis referring to a gum condition that is not accompanied by the destruction of the alveolar bone around the tooth and periodontitis being a gum condition accompanied by the destruction of the alveolar bone. The concentration criteria thereof may be used as a concentration to distinguish a healthy state and the existence of a disease.
\textcircled{1} Bacteria with AUC greater than or equal to 0.7
\textcircled{2} 16S rRNA sequencing (%): Total bacteria
\textcircled{3} RT-PCR (%): P. gingivalis, T. forsythia, P. intermedia, P. endodontalis, F. alocis
\textcircled{4} RT-PCR (count): P. gingivalis, T. forsythia, T. denticola, P. intermedia, P. endodontalis, F. alocis, P. micra

### (Example) Concentration of Bacteria as a Standard for Diagnosing Periodontitis Using Saliva and a Diagnosis Method Thereof

In the case of P. gingivalis, when the concentration of the bacteria in saliva is measured by real-time PCR using the primer shown in Table 2, if the % of P. gingivalis compared to the total bacteria is less than or equal to 0.0034% while being greater than 0.0034% in healthy gums, the AUC of those diagnosed as gums with diseases (gingivitis and periodontitis) is 0.79, indicating that the standard of 0.0034% is a way to distinguish between healthy gums and diseases (gingivitis and periodontitis). It has a sensitivity of 0.83 and a specificity of 0.75.

TABLE 6

| | | cutoff | AUC | sen | spe |
|---|---|---|---|---|---|
| NGS (%) | P. gingivalis | 0.5276 | 0.75 | 0.50 | 1.00 |
| | T. forsythia | 0.0352 | 0.73 | 0.72 | 0.75 |
| | T. denticola | 0.0044 | 0.70 | 0.77 | 0.63 |
| | P. intermedia | 0.0079 | 0.73 | 0.70 | 0.75 |
| | P. endodontalis | 0.1662 | 0.83 | 0.66 | 1.00 |
| | F. alocis | 0.0141 | 0.82 | 0.77 | 0.88 |
| | F. nucleatum | 1.4741 | 0.76 | 0.64 | 0.88 |
| | P. micra | 0.1722 | 0.72 | 0.44 | 1.00 |
| | R. dentocariosa | 0.6632 | 0.72 | 0.19 | 0.38 |
| RT-PCR (%) | P. gingivalis | 0.0034 | 0.79 | 0.83 | 0.75 |
| | T. forsythia | 0.0021 | 0.77 | 0.78 | 0.75 |
| | T. denticola | 0.0000 | 0.69 | 0.88 | 0.50 |
| | P. intermedia | 0.0013 | 0.71 | 0.80 | 0.63 |
| | P. endodontalis | 0.0062 | 0.80 | 0.59 | 1.00 |
| | F. alocis | 0.0004 | 0.86 | 0.84 | 0.88 |
| | F. nucleatum | 0.2401 | 0.63 | 0.48 | 0.25 |
| | P. micra | 0.0077 | 0.67 | 0.97 | 0.38 |
| | R. dentocariosa | 0.0269 | 0.66 | 0.31 | 0.38 |
| RT-PCR (count) | P. gingivalis | 5 | 0.82 | 0.89 | 0.75 |
| | T. forsythia | 10 | 0.75 | 0.63 | 0.88 |
| | T. denticola | 11 | 0.73 | 0.58 | 0.88 |
| | P. intermedia | 1 | 0.71 | 0.80 | 0.63 |
| | P. endodontalis | 9 | 0.82 | 0.64 | 1.00 |
| | F. alocis | 5 | 0.88 | 0.89 | 0.88 |
| | F. nucleatum | 1036 | 0.68 | 0.36 | 1.00 |
| | P. micra | 94 | 0.78 | 0.94 | 0.63 |
| | R. dentocariosa | 44 | 0.56 | 0.50 | 0.38 |

### (2) (Healthy Gums, Gingivitis) vs. (Moderate Periodontitis, Severe Periodontitis)

It is a method of distinguishing healthy gums and gingivitis from periodontitis (moderate or severe). In other words, the distinction between a state without bone loss and a state with bone loss is meaningful because gingivitis may be reversibly restored to its original state by treatment, but periodontitis is a condition accompanied by irreversible destruction that cannot be restored to its original state by treatment.
\textcircled{1} Bacteria with AUC greater than or equal to 0.7
\textcircled{2} 16S rRNA sequencing (%): P. gingivalis, T. forsythia, P. intermedia, P. endodontalis, F. alocis, F. nucleatum, P. micra, R. dentocariosa
\textcircled{3} RT-PCR (%): P. gingivalis, T. forsythia
\textcircled{4} RT-PCR (count): P. gingivalis, T. forsythia, T. denticola, P. intermedia, P. endodontalis, F. alocis, F. nucleatum, P. micra

### (Example) Concentration of Bacteria as the Standard for Diagnosing Periodontitis Using Saliva and a Diagnosis Method Thereof

In the case of P. gingivalis, when the concentration of the bacteria in saliva is measured by real-time PCR using the primer shown in Table 2, if the % of P. gingivalis compared to the total bacteria is less than or equal to 0.0586% while being greater than 0.0034% in healthy gums or gums with gingivitis, the AUC of those diagnosed as gums with periodontitis is 0.73, indicating that the standard of 0.0586% is a way to distinguish the healthy gums and gums with gingivitis without destruction of alveolar bone from gums with periodontitis accompanied by destruction of alveolar bone. It has a sensitivity of 0.71 and a specificity of 0.75.

TABLE 7

| | var | cutoff | AUC | sen | spe |
|---|---|---|---|---|---|
| NGS (%) | P. gingivalis | 0.2258 | 0.77 | 0.71 | 0.83 |
| | T. forsythia | 0.0611 | 0.74 | 0.73 | 0.75 |
| | T. denticola | 0.0841 | 0.68 | 0.40 | 0.96 |
| | P. intermedia | 0.1594 | 0.71 | 0.58 | 0.83 |
| | P. endodontalis | 0.7348 | 0.72 | 0.48 | 0.96 |
| | F. alocis | 0.1141 | 0.76 | 0.56 | 0.96 |
| | F. nucleatum | 1.2996 | 0.72 | 0.77 | 0.67 |
| | P. micra | 0.2103 | 0.70 | 0.48 | 0.92 |
| | R. dentocariosa | 0.3136 | 0.71 | 0.29 | 0.29 |
| RT-PCR (%) | P. gingivalis | 0.0586 | 0.73 | 0.71 | 0.75 |
| | T. forsythia | 0.0048 | 0.71 | 0.75 | 0.67 |
| | T. denticola | 0.0113 | 0.66 | 0.52 | 0.79 |
| | P. intermedia | 0.0037 | 0.68 | 0.73 | 0.63 |
| | P. endodontalis | 0.0074 | 0.63 | 0.58 | 0.67 |
| | F. alocis | 0.0062 | 0.68 | 0.60 | 0.75 |
| | F. nucleatum | 0.2370 | 0.56 | 0.50 | 0.38 |
| | P. micra | 1.1486 | 0.61 | 0.27 | 0.96 |
| | R. dentocariosa | 0.0269 | 0.68 | 0.23 | 0.42 |
| RT-PCR (count) | P. gingivalis | 33 | 0.78 | 0.81 | 0.75 |
| | T. forsythia | 7 | 0.77 | 0.79 | 0.75 |
| | T. denticola | 11 | 0.77 | 0.71 | 0.83 |
| | P. intermedia | 3 | 0.71 | 0.79 | 0.63 |
| | P. endodontalis | 29 | 0.71 | 0.58 | 0.83 |
| | F. alocis | 16 | 0.75 | 0.83 | 0.67 |
| | F. nucleatum | 529 | 0.71 | 0.67 | 0.75 |
| | P. micra | 233 | 0.74 | 0.90 | 0.58 |
| | R. dentocariosa | 11 | 0.57 | 0.81 | 0.33 |

### (3) (Healthy Gums, Gingivitis, Moderate Periodontitis) vs. (Severe Periodontitis)

It is a method of distinguishing healthy gums, gingivitis, and moderate periodontitis from severe periodontitis. Severe periodontitis is a severe condition in which partial loss of alveolar bone is severe with probability of the tooth loss, and thus clinical distinction thereof is essential. If a point-of-care diagnostic kit is developed based on the concentration of the bacteria, it may be said that the concentration standard indicates a condition that the subject must visit the dentist.
\textcircled{1} Bacteria with AUC greater than or equal to 0.7
\textcircled{2} 16S rRNA sequencing (%): P. gingivalis. T. forsythia, P. intermedia, P. endodontalis, F. alocis, F. nucleatum
\textcircled{3} RT-PCR (%): P. gingivalis. T. forsythia, P. intermedia,
\textcircled{4} RT-PCR (count): P. gingivalis. T. forsythia. T. denticola, P. intermedia, F.

alocis. F. nucleatum

### (Example) Concentration of Bacteria as the Standard for Diagnosing Periodontitis Using Saliva and a Diagnosis Method Thereof

In the case of P. gingivalis, when the concentration of the bacteria in saliva is measured by real-time PCR using the primer shown in Table 2, if the % of P. gingivalis 15 compared to the total bacteria is less than or equal to 0.1122% while being greater than 0.1122% in healthy gums or gums with gingivitis or moderate periodontitis, it may be the gum with severe periodontitis, such that the diagnosis as the severe periodontitis may be an indicator of the time to visit the dentist as soon as possible, wherein the AUC of diagnosis is 0.74, indicating that the standard of 0.1122% is a way to distinguish healthy gums and gums with gingivitis 20 and moderate periodontitis without destruction of alveolar bone from gums with severe periodontitis that is accompanied by severe destruction of alveolar bone and requires immediate treatment. It has a sensitivity of 0.76 and a specificity of 0.72.

TABLE 8

| | var | cutoff | AUC | sen | spe |
|---|---|---|---|---|---|
| NGS (%) | P. gingivalis | 1.4374 | 0.82 | 0.72 | 0.91 |
| | T. forsythia | 0.1443 | 0.79 | 0.79 | 0.79 |
| | T. denticola | 0.2061 | 0.66 | 0.41 | 0.91 |
| | P. intermedia | 0.0294 | 0.81 | 0.97 | 0.65 |
| | P. endodontalis | 0.7348 | 0.74 | 0.62 | 0.86 |
| | F. alocis | 0.1141 | 0.81 | 0.76 | 0.86 |
| | F. nucleatum | 1.8720 | 0.77 | 0.83 | 0.72 |
| | P. micra | 0.1197 | 0.68 | 0.69 | 0.67 |
| | R. dentocariosa | 0.2403 | 0.53 | 0.55 | 0.51 |
| RT-PCR (%) | P. gingivalis | 0.1122 | 0.74 | 0.76 | 0.72 |
| | T. forsythia | 0.0297 | 0.74 | 0.62 | 0.86 |
| | T. denticola | 0.0125 | 0.61 | 0.52 | 0.70 |
| | P. intermedia | 0.0037 | 0.74 | 0.90 | 0.58 |
| | P. endodontalis | 0.0329 | 0.60 | 0.48 | 0.72 |
| | F. alocis | 0.0267 | 0.66 | 0.41 | 0.91 |
| | F. nucleatum | 0.2401 | 0.55 | 0.45 | 0.44 |
| | P. micra | 1.1486 | 0.57 | 0.28 | 0.86 |
| | R. dentocariosa | 0.0167 | 0.66 | 0.28 | 0.40 |
| RT-PCR (count) | P. gingivalis | 838 | 0.78 | 0.72 | 0.84 |
| | T. forsythia | 213 | 0.80 | 0.62 | 0.98 |
| | T. denticola | 25 | 0.71 | 0.66 | 0.77 |
| | P. intermedia | 18 | 0.80 | 0.86 | 0.74 |
| | P. endodontalis | 79 | 0.69 | 0.59 | 0.79 |
| | F. alocis | 159 | 0.73 | 0.69 | 0.77 |
| | F. nucleatum | 529 | 0.72 | 0.79 | 0.65 |
| | P. micra | 891 | 0.69 | 0.76 | 0.63 |
| | R. dentocariosa | 215 | 0.59 | 0.28 | 0.91 |

5) A Method of Utilizing Saliva Samples for the Disease Severity Based on Equations Obtained from 16S rRNA Sequencing Analysis and Real-Time PCR Analysis

Using the concentration of 9 species of bacteria in saliva, the severity of the periodontal disease in the oral cavity from which the sample was collected may be distinguished into healthy gums, gingivitis, moderate periodontitis, and severe periodontitis by the Formulas calculated below.

1. A Method of Utilizing Concentrations of 9 Species of Bacteria Obtained Through the Method of 16S rRNA Sequencing

There are four Formulas that yield healthy gums, gums with gingivitis, gums with moderate periodontitis, and gums with severe periodontitis according to the severity of the periodontal disease.

$$Formula for healthy gums = - 3.52072 + ( - 0.026 \; 39 ) \times ( P . gingivalis \% ) + ( 0.839493 ) \times ( T . forsythia \% ) + ( 0.316224 ) \times ( T . denticola \% ) + ( - 0.986 \; 41 ) \times ( F . alocis \% ) + ( 0.646425 ) \times ( P . intermedia \% ) + ( 0.087085 ) \times ( P . endodontalis \% ) + ( - 0.087 \; 49 ) \times ( F . nucleatum \% ) + ( 1.391072 ) \times ( R . dentocariosa \% ) + ( 0.39933 ) \times ( P . micra \% ) \qquad ( 1 )$$

$$Formula for gums with gingivitis = - 1.9748 + ( - 0.011 \; 37 ) \times ( P . gingivalis \% ) + ( - 0.96119 ) \times ( T . forsythia \% ) + ( 0.0033 \; 46 ) \times ( T . denticola \% ) + ( - 0.25645 ) \times ( F . alocis \% ) + ( 0.568448 ) \times ( P . intermedia \% ) + ( 0.42965 ) \times ( P . endodontalis \% ) + ( 0.2009 \; 52 ) \times ( F . nucleatum \% ) + ( 0.519156 ) \times ( R . dentocariosa \% ) + ( - 0.52904 ) \times ( P . micra \% ) \qquad ( 2 )$$

$$Formula for gums with moderate periodontitis = - 1.9075 + ( 0.0070 \; 68 ) \times ( P . gingivalis \% ) + ( 1.141592 ) \times ( T . forsythia \% ) + ( - 0.2656 ) \times ( T . denticola \% ) + ( - 1.46872 ) \times ( F . alocis \% ) + ( 0.715288 ) \times ( P . intermedia \% ) + ( 0.77475 ) \times ( P . endodontalis \% ) + ( - 0.04183 ) \times ( F . nucleatum \% ) + ( 0.169773 ) \times ( R . dentocariosa \% ) + ( 1.520645 ) \times ( P . micra \% ) \qquad ( 3 )$$

$$Formula for gums with severe periodontitis = - 3.24579 + ( 0.296464 ) \times ( P . gingivalis \% ) + ( - 0.1594 ) \times ( T . forsythia \% ) + ( - 2.53253 ) \times ( T . denticola \% ) + ( - 0.11511 ) \times ( F . alocis \% ) + ( 1.83945 ) \times ( P . intermedia \% ) + ( 1.407898 ) \times ( P . endodontalis \% ) + ( 0.248094 ) \times ( F . nucleatum \% ) + ( 0.300277 ) \times ( R . dentocariosa \% ) + ( - 0.74747 ) \times ( P . micra \% ) \qquad ( 4 )$$

After collecting saliva of an individual of interest to know the severity of the disease and then performing 16S rRNA sequencing on the bacteria present in the saliva, the concentration % of the bacteria relative to the total bacteria for the 9 bacterial species may be calculated and then the % of the 9 bacterial species calculated may be applied to 4 Formulas to determine the severity of gum disease corresponding to the Formula with the highest value as the severity of disease in the oral cavity.

When applying % for each of the 9 bacterial species derived from 16S IRNA sequencing to corresponding Formula for the analyzed saliva samples from 8 people with healthy gums, 16 people with gingival gums, 19 people with moderate periodontitis, and 29 people with severe periodontitis

Of the 8 samples of healthy gums, 2 samples (25%) were analyzed as healthy gums, 4 samples (50%) as gums with gingivitis, and 2 samples (25%) as moderate periodontitis.

Of the 16 samples of gums with gingivitis, 8 samples (50%) were analyzed as gums with gingivitis, 1 sample (6.25%) as healthy gums, 5 samples (31.25%) as gums with moderate periodontitis, and 2 samples (12.50%) as severe periodontitis.

Of the 19 samples of gums with moderate periodontitis, 13 samples (68.42%) were analyzed as gums with moderate periodontitis, 2 samples (10.53%) as gums with gingivitis, and 4 samples (21.05%) as severe periodontitis.

Of the 29 samples of gums with severe periodontitis, 21 samples (72.41%) were analyzed as gums with severe periodontitis, 1 sample (3.45%) as healthy gums, 3 samples (10.34%) as gums with gingivitis, and 4 samples (13.79%) as gums with moderate periodontitis.

### 2. Method of Utilizing the Bacterial % of 9 Bacterial Species Obtained Through the Real-Time PCR Method Compared to the Total Bacteria

There are four Formulas that yield healthy gums, gums with gingivitis, gums with moderate periodontitis, and gums with severe periodontitis according to the severity of the periodontal disease.

$$Formula for healthy gums = - 3.60258 + ( - 0.051 ) \times ( P . gingivalis \% ) + ( 3.457539 ) \times ( T . forsythia \% ) + ( - 10.0746 ) \times ( T . denticola \% ) + ( - 1.25858 ) \times ( F . alocis \% ) + ( - 1.7208 ) \times ( P . intermedia \% ) + ( - 8.5888 ) \times ( P . endodontalis \% ) + ( 1.976449 ) \times ( F . nucleatum \% ) + ( 5.348558 ) \times ( R . dentocariosa \% ) + ( 0.063522 ) \times ( P . micra \% ) \qquad ( 1 )$$

$$Formula for gums with gingivitis = - 1.8156 + ( - 0.00877 ) \times ( P . gingivalis \% ) + ( 5.590504 ) \times ( T . forsythia \% ) + ( - 4.75712 ) \times ( T . denticola \% ) + ( - 1.4037 ) \times ( F . alocis \% ) + ( - 4.88342 ) \times ( P . intermedia \% ) + ( 5.253621 ) \times ( P . endodontalis \% ) + ( 0.331331 ) \times ( F . nucleatum \% ) + ( 3.02979 ) \times ( R . dentocariosa \% ) + ( 0.152853 ) \times ( P . micra \% ) \qquad ( 2 )$$

$$Formula for gums with moderate periodontitis = - 2.18027 + ( 0.14454 ) \times ( P . gingivalis \% ) + ( 12.54856 ) \times ( T . forsythia \% ) + ( 0.984939 ) \times ( T . denticola \% ) + ( - 4.98201 ) \times ( F . alocis \% ) + ( - 6.46175 ) \times ( P . intermedia \% ) + ( 3.469277 ) \times ( P . endodontalis \% ) + ( 1.076105 ) \times ( F . nucleatum \% ) + ( - 1.23915 ) \times ( R . dentocariosa \% ) + ( 0.468393 ) \times ( P . micra \% ) \qquad ( 3 )$$

$$Formula for gums with severe periodontitis = - 1.84498 + ( - 0.00562 ) \times ( P . gingivalis \% ) + ( 13.76487 ) \times ( T . forsythia \% ) + ( 2.553924 ) \times ( T . denticola \% ) + ( 0.054474 ) \times ( F . alocis \% ) + ( 7.646578 ) \times ( P . intermedia \% ) + ( 4.540174 ) \times ( P . endodontalis \% ) + ( - 0.48413 ) \times ( F . nucleatum \% ) + ( - 0.52195 ) \times ( R . dentocariosa \% ) + ( 0.21387 ) \times ( P . micra \% ) \qquad ( 4 )$$

After collecting saliva from an individual of interest to know the severity of the disease and then performing real-time PCR on the bacteria present in the saliva using bacteria-specific primers, the concentration % of the bacteria relative to the total bacteria for the 9 bacterial species may be calculated and then the bacterial % of the 9 species calculated may be applied to the 4 Formulas to determine the severity of gum disease corresponding to the Formula with the highest value as the severity of disease in the oral cavity.

When applying % for each of the 9 bacterial species obtained from real-time PCR to corresponding Formula for the analyzed saliva samples from 8 people with healthy gums, 16 people with gingival gums, 19 people with moderate periodontitis, and 29 people with severe periodontitis

Of the 8 samples of healthy gums, 3 samples (37.50%) were analyzed as healthy gums, 4 samples (50%) as gums with gingivitis, and 1 sample (12.50%) as severe periodontitis.

Of the 16 samples of gums with gingivitis, 7 samples (43.75%) were analyzed as gums with gingivitis, 3 samples (18.75%) as gums with moderate periodontitis, and 6 samples (37.50%) as gums with severe periodontitis.

Of the 19 samples of gums with moderate periodontitis, 8 samples (42.11%) were analyzed as gums with moderate periodontitis, 6 samples (31.58%) as gums with gingivitis, and 5 samples (26.32%) as severe periodontitis.

Of the 29 samples of gums with severe periodontitis, 24 samples (82.76%) were analyzed as gums with severe periodontitis, and 5 samples (17.24%) as gums with gingivitis.

### 3. Method of Utilizing the Bacterial Count of 9 Species Obtained Through the Real-Time PCR Method

There are four Formulas that yield healthy gums, gums with gingivitis, gums with moderate periodontitis, and gums with severe periodontitis according to the severity of the periodontal disease.

$$Formula for healthy gums = - 2.30476 + ( - 1.49793 ) \times ( P . gingivalis count ) + ( 42.24108 ) \times ( T . forsythia count ) + ( - 51.0784 ) \times ( T . denticola count ) + ( 0.039624 ) \times ( F . alocis count ) + ( - 26.4862 ) \times ( P . intermedia count ) + ( - 90.5605 ) \times ( P . endodontalis count ) + ( 39.28479 ) \times ( F . nucleatum count ) + ( 17.65564 ) \times ( R . dentocariosa count ) + ( 4.278468 ) \times ( P . micra count ) \qquad ( 1 )$$

$$Formula for gums with gingivitis = - 1.62912 + ( - 0.76032 ) \times ( P . gingivalis count ) + ( 50.56172 ) \times ( T . forsythia count ) + ( - 65.645 ) \times ( T . denticola count ) + ( - 7.76311 ) \times ( F . alocis count ) + ( - 47.378 ) \times ( P . intermedia count ) + ( 45.18652 ) \times ( P . endodontalis count ) + ( 26.42666 ) \times ( F . nucleatum count ) + ( 47.9035 ) \times ( R . dentocariosa count ) + ( 5.394878 ) \times ( P . micra count ) \qquad ( 2 )$$

$$Formula for gums with moderate periodontitis = - 1.8899 + ( - 2.13304 ) \times ( P . gingivalis count ) + ( 121.1973 ) \times ( T . forsythia count ) + ( - 123.48 ) \times ( T . denticola count ) + ( - 11.1651 ) \times ( F . alocis count ) + ( - 83.2768 ) \times ( P . intermedia count ) + ( - 64.0856 ) \times ( P . endodontalis count ) + ( 43.52085 ) \times ( F . nucleatum count ) + ( 24.10447 ) \times ( R . dentocariosa count ) + ( 32.03642 ) \times ( P . micra count ) \qquad ( 3 )$$

$$Formula for gums with severe periodontitis = - 2.79975 + ( - 0.26374 ) \times ( P . gingivalis count ) + ( 366.5963 ) \times ( T . forsythia count ) + ( - 147.396 ) \times ( T . denticola count ) + ( 39.73937 ) \times ( F . alocis count ) + ( - 140.134 ) \times ( P . intermedia count ) + ( - 102.176 ) \times ( P . endodontalis count ) + ( 55.15521 ) \times ( F . nucleatum count ) + ( 123.9248 ) \times ( R . dentocariosa count ) + ( 29.36675 ) \times ( P . micra count ) \qquad ( 4 )$$

After collecting saliva of an individual of interest to know the severity of the disease and then performing real-time PCR on the bacteria present in the saliva using bacteria-specific primers, the bacterial count for 9 bacterial species may be calculated and then the calculated bacterial count of the 9 species may be applied to 4 Formulas to determine the severity of gum disease corresponding to the Formula with the highest value as the severity of disease in the oral cavity.

When applying bacterial count for each of the 9 bacterial species obtained from real-time PCR to corresponding Formula for the analyzed saliva samples from 8 people with healthy gums, 16 people with gingival gums, 19 people with moderate periodontitis, and 29 people with severe periodontitis

None of the 8 samples with healthy gums were classified as healthy gums, and 6 samples (75%) were analyzed as gums with gingivitis and 2 samples (25%) as moderate periodontitis.

Of the 16 samples of gums with gingivitis, 13 samples (81.25%) were analyzed as gums with gingivitis, 1 sample (6.25%) as gums with moderate periodontitis, and 2 samples (12.50%) as gums with severe periodontitis.

Of the 19 samples of gums with moderate periodontitis, 8 samples (42.11%) were analyzed as gums with moderate periodontitis, 9 samples (47.37%) as gums with gingivitis, and 2 samples (10.53%) as severe periodontitis.

Of the 29 samples of gums with severe periodontitis, 18 samples (62.07%) were analyzed as gums with severe periodontitis, 4 samples (13.79%) as gums with gingivitis, and 7 samples (24.14%) as moderate periodontitis.

Having described specific parts of the present disclosure in detail above, it is clear to those skilled in the art that these specific descriptions are merely preferred example embodiments and do not limit the scope of the present disclosure. Thus, the substantial scope of the present disclosure will be defined by the appended claims and their equivalents.

The scope of the present disclosure is indicated by the claims described below, and all changes or modified forms derived from the meaning and scope of the claims and their equivalent concepts should be construed as being included in the scope of the present disclosure.

1. A method of providing information necessary for diagnosis of a periodontal disease, the method comprising:
performing quantitative analysis by real-time polymerase chain reaction (real-time PCR) with one or more bacteria selected from the group consisting of Porphyromonas gingivalis, Tannerella forsythia, Treponema denticola, Prevotella intermedia, Porphyromonas endodontalis, and Filifactor alocis, Fusobacterium micleatum, and Parvimonas micra in a sample isolated from an individual; and
comparing a bacterial % or bacterial count obtained through the quantitative analysis by real-time PCR with that selected from the group consisting of normal individuals and patients with gingivitis, moderate periodontitis, and severe periodontitis.

2. (canceled)

3. The method of claim 1, wherein the comparing comprises comparing the bacterial % or bacterial count obtained through the quantitative analysis by real-time PCR with a cut-off value set through the bacterial % and the bacterial count according to severity of the disease divided by a probing pocket, a degree of bleeding on probing, and a degree of alveolar bone resorption which indicate the severity of periodontitis.

4. The method of claim 1, wherein the sample is saliva.

5. The method of claim 1, wherein the quantitative analysis by real-time PCR measures an expression level of a target gene using any one or more primer sets selected from the group consisting of a primer set represented by SEQ ID NOS: 1 and 2, a primer set represented by SEQ ID NOS: 3 and 4, a primer set represented by SEQ ID NOS: 5 and 6, a primer set represented by SEQ ID NOS: 7 and 8, a primer set represented by SEQ ID NOS: 9 and 10, a primer set represented by SEQ ID NOS: 11 and 12, a primer set represented by SEQ ID NOS: 13 and 14, and a primer set represented by SEQ ID NOS: 15 and 16.

6. The method of claim 5, wherein the target gene of the primer set represented by SEQ ID NOS: 1 and 2 is rpoB in Porphyromonas gingivalis, the target gene of the primer set represented by SEQ ID NOS: 3 and 4 is rpoB in Tannerella forsythia, the target gene of the primer set represented by SEQ ID NOS: 5 and 6 is rpoB in Prevotella intermedia, the target gene of the primer set represented by SEQ ID NOS: 7 and 8 is rpoB in Porphyromonas endodontalis, the target gene of the primer set represented by SEQ ID NOS: 9 and 10 is 16s rRNA in Filifactor alocis, the target gene of the primer set represented by SEQ ID NOS: 11 and 12 is rpoB in Treponema denticola, the target gene of the primer set represented by SEQ ID NOS: 13 and 14 is rpoB in Fusobacterium micleatum, and the target gene of the primer set represented by SEQ ID NOS: 15 and 16 is fusA in Parvimonas micra.

7. The method of claim 3, wherein the comparing comprises distinguishing healthy gums and the periodontal disease if, based on a cut-off value of the bacterial % obtained through the quantitative analysis by real-time PCR, the cut-off value of the target gene of the primer set represented by SEQ ID NOS: 1 and 2 is greater than or equal to 0.0034, the cut-off value of the target gene of the primer set represented by SEQ ID NOS: 3 and 4 is greater than or equal to 0.0021, the cut-off value of the target gene of the primer set represented by SEQ ID NOS: 5 and 6 is greater than or equal to 0.0013, the cut-off value of the target gene of the primer set represented by SEQ ID NOS: 7 and 8 is greater than or equal to 0.0062, or the cut-off value of the target gene of the primer set represented by SEQ ID NOS: 9 and 10 is greater than or equal to 0.0004.

8. The method of claim 3, wherein the comparing comprises distinguishing healthy gum and the periodontal disease if, based on a cut-off value of the bacterial count obtained through the quantitative analysis by real-time PCR, the cut-off value of the target gene of the primer set represented by SEQ ID NOS: 1 and 2 is greater than or equal to 5, the cut-off value of the target gene of the primer set represented by SEQ ID NOS: 3 and 4 is greater than or equal to 10, the cut-off value of the target gene of the primer set represented by SEQ ID NOS: 5 and 6 is greater than or equal to 1, the cut-off value of the target gene of the primer set represented by SEQ ID NOS: 7 and 8 is greater than or equal to 9, the cut-off value of the target gene of the primer set represented by SEQ ID NOS: 9 and 10 is greater than or equal to 5, the cut-off value of the target gene of the primer set represented by SEQ ID NOS: 11 and 12 is greater than or equal to 11, or the cut-off value of the target gene of the primer set represented by SEQ ID NOS: 15 and 16 is greater than or equal to 94.

9. The method of claim 3, wherein the cut-off value is derived from a cut-off value of bacteria with an AUC value greater than or equal to 0.7 by obtaining an ROC curve and the AUC value to distinguish the healthy gums and gums with the periodontal disease based on the bacterial % or bacterial count from a saliva sample compared to the total bacteria of salivary bacteria collected from the oral cavity of the individual, and wherein the periodontal disease comprises gingivitis, which is defined by a subject whose probing pocket depth of individual teeth is less than or equal to 3 mm and degree of bleeding on probing of an entire dentition is greater than or equal to 10% and periodontitis which is accompanied by alveolar bone resorption.

10. (canceled)

11. The method of claim 3, wherein the comparing comprises distinguishing healthy gum/gingivitis and periodontitis if, based on the cut-off value of the bacterial % obtained through the quantitative analysis by real-time PCR, the cut-off value of the target gene of the primer set represented by SEQ ID NOS: 1 and 2 is greater than or equal to 0.0586, or the cut-off value of the target gene of the primer set represented by SEQ ID NOS: 3 and 4 is greater than or equal to 0.0048.

12. The method of claim 3, wherein the comparing comprises distinguishing healthy gum/gingivitis and periodontitis if, based on a cut-off value of the bacterial count obtained through the quantitative analysis by real-time PCR, the cut-off value of the target gene of the primer set represented by SEQ ID NOS: 1 and 2 is greater than or equal to 33, the cut-off value of the target gene of the primer set represented by SEQ ID NOS: 3 and 4 is greater than or equal to 7, the cut-off value of the target gene of the primer set represented by SEQ ID NOS: 5 and 6 is greater than or equal to 3, the cut-off value of the target gene of the primer set represented by SEQ ID NOS: 7 and 8 is greater than or equal to 29, the cut-off value of the target gene of the primer set represented by SEQ ID NOS: 9 and 10 is greater than or equal to 16, the cut-off value of the target gene of the primer set represented by SEQ ID NOS: 11 and 12 is greater than or equal to 11, the cut-off value of the target gene of the primer set represented by SEQ ID NOS: 13 and 14 is greater than or equal to 529, or the cut-off value of the target gene of the primer set represented by SEQ ID NOS: 15 and 16 is greater than or equal to 233.

13. The method of claim 3, wherein the cut-off value is derived from a cut-off value of bacteria with an AUC value greater than or equal to 0.7 by obtaining an ROC curve and the AUC value to distinguish gums with periodontitis accompanied by alveolar bone resorption from healthy gums and gums with gingivitis in a gum state not accompanied by alveolar bone resorption based on the bacterial % or bacterial count from a saliva sample compared to the total bacteria of salivary bacteria collected from the oral cavity of the individual.

14. (canceled)

15. The method of claim 3, wherein the comparing comprises distinguishing healthy gum/gingivitis/moderate periodontitis and severe periodontitis if, based on a cut-off value of the bacterial % obtained through the quantitative analysis by real-time PCR, the cut-off value of the target gene of the primer set represented by SEQ ID NOS: 1 and 2 is greater than or equal to 0.1122, the cut-off value of the target gene of the primer set represented by SEQ ID NOS: 3 and 4 is greater than or equal to 0.0297, or the cut-off value of the target gene of the primer set represented by SEQ ID NOS: 5 and 6 is greater than or equal to 0.0037.

16. The method of claim 3, wherein the comparing comprises distinguishing healthy gum/gingivitis/moderate periodontitis and severe periodontitis if, based on a cut-off value of the bacterial count obtained through the quantitative analysis by real-time PCR, the cut-off value of the target gene of the primer set represented by SEQ ID NOS: 1 and 2 is greater than or equal to 838, the cut-off value of the target gene of the primer set represented by SEQ ID NOS: 3 and 4 is greater than or equal to 213, the cut-off value of the target gene of the primer set represented by SEQ ID NOS: 5 and 6 is greater than or equal to 18, the cut-off value of the target gene of the primer set represented by SEQ ID NOS: 9 and 10 is greater than or equal to 159, or the cut-off value of the target gene of the primer set represented by SEQ ID NOS: 13 and 14 is greater than or equal to 529.

17. The method of claim 3, wherein the cut-off value is derived from a cut-off value of bacteria with an AUC value greater than or equal to 0.7 by obtaining an ROC curve and the AUC value to distinguish the healthy gums, gums with gingivitis and gums with moderate periodontitis from gums with severe periodontitis based on the bacterial % or bacterial count from a saliva sample compared to the total bacteria of salivary bacteria collected from the oral cavity of the individual, and wherein the severe periodontitis comprises periodontitis that shows probing pockets with a probing pocket depth greater than or equal to 6 mm locally and vertical bone resorption greater than or equal to 3 mm and has furcation-involved lesions caused by alveolar bone resorption in posterior teeth.

18. (canceled)

19. A method of providing information necessary for diagnosis of a periodontal disease, the method comprising:
performing 16S rRNA sequencing analysis with one or more bacteria selected from the group consisting of Porphyromonas gingivalis, Tannerella forsythia, Treponema denticola, Prevotella intermedia, Porphyromonas endodontalis, Filifactor alocis, Fusobacterium nucleatum, and Parvimonas micra in a sample isolated from an individual; and
comparing a bacterial % obtained through the 16S rRNA sequencing analysis with that selected from the group consisting of normal individuals and patients with gingivitis, moderate periodontitis, and severe periodontitis.

20. The method of claim 19, wherein cut-off values are 0.5276 for Porphyromonas gingivalis, 0.0352 for Tannerella forsythia, 0.0044 for Treponema denticola, 0.0079 for Prevotella intermedia, 0.1662 for Porphyromonas endodontalis, 0.0141 for Filifactor alocis, 1.4741 for Fusobacterium nucleatum, 0.1722 for Parvimonas micra, or 0.6632 for Rothia dentocariosa, and, if the bacterial % is greater than or equal to the corresponding cut-off value, healthy gums and periodontal diseases are distinguished.

21. The method of claim 19, wherein cut-off values are 0.2258 for Porphyromonas gingivalis, 0.0611 for Tannerella forsythia, 0.1594 for Prevotella intermedia, 0.7348 for Porphyromonas endodontalis, 0.1141 for Filifactor alocis, 1.2996 for Fusobacterium nucleatum, 0.2103 for Parvimonas micra, or 0.3136 for Rothia dentocariosa, and, if the bacterial % is greater than or equal to the corresponding cut-off value, healthy gum/gingivitis and periodontitis are distinguished.

22. The method of claim 19, wherein cut-off values are 1.4374 for Porphyromonas gingivalis, 0.1443 for Tannerella forsythia, 0.0294 for Prevotella intermedia, 0.7348 for Porphyromonas endodontalis, 0.1141 for Filifactor alocis, or 1.8720 for Fusobacterium nucleatum, and, if the bacterial % is greater than or equal to the corresponding cut-off value, healthy gum/gingivitis/moderate periodontitis and severe periodontitis are distinguished.

23. The method of claim 19, wherein the sample is saliva.

24-26. (canceled)

27-28. (canceled)


SEQUENCE LISTING (20 sequences)

SEQ ID NO: 1 - DNA, 25 bp, Porphyromonas gingivalis
  source 1..25: mol_type = genomic DNA
ggaagagaagaccgtagcacaagga

SEQ ID NO: 2 - DNA, 25 bp, Porphyromonas gingivalis
  source 1..25: mol_type = genomic DNA
gagtaggcgaaacgtccatcaggtc

SEQ ID NO: 3 - DNA, 22 bp, Porphyromonas gingivalis
  source 1..22: mol_type = genomic DNA
ggattgaccaccggcgaagaca

SEQ ID NO: 4 - DNA, 24 bp, Porphyromonas gingivalis
  source 1..24: mol_type = genomic DNA
cggacacgacggttactcaaatgg

SEQ ID NO: 5 - DNA, 20 bp, Porphyromonas gingivalis
  source 1..20: mol_type = genomic DNA
acccattggcagaagttacg

SEQ ID NO: 6 - DNA, 24 bp, Porphyromonas gingivalis
  source 1..24: mol_type = genomic DNA
tcaataggacacaagcgaccatag

SEQ ID NO: 7 - DNA, 24 bp, Porphyromonas gingivalis
  source 1..24: mol_type = genomic DNA
agcagagttccgtcgtcgtattca

SEQ ID NO: 8 - DNA, 20 bp, Porphyromonas gingivalis
  source 1..20: mol_type = genomic DNA
gcgccctgccatcttgtctc

SEQ ID NO: 9 - DNA, 20 bp, Porphyromonas gingivalis
  source 1..20: mol_type = genomic DNA
aaccggagcaaaactgagaa

SEQ ID NO: 10 - DNA, 20 bp, Porphyromonas gingivalis
  source 1..20: mol_type = genomic DNA
ccgtccgccactaacttcta

SEQ ID NO: 11 - DNA, 23 bp, Porphyromonas gingivalis
  source 1..23: mol_type = genomic DNA
cgggcgtgcatcttgtcgtctac

SEQ ID NO: 12 - DNA, 22 bp, Porphyromonas gingivalis
  source 1..22: mol_type = genomic DNA
cttaaccggccgcctctttgaa

SEQ ID NO: 13 - DNA, 22 bp, Porphyromonas gingivalis
  source 1..22: mol_type = genomic DNA
acctaagggagaaacagaacca

SEQ ID NO: 14 - DNA, 22 bp, Porphyromonas gingivalis
  source 1..22: mol_type = genomic DNA
cctgcctttaattcatctccat

SEQ ID NO: 15 - DNA, 23 bp, Porphyromonas gingivalis
  source 1..23: mol_type = genomic DNA
agaatcaatttctcaaggtgcag

SEQ ID NO: 16 - DNA, 24 bp, Porphyromonas gingivalis
  source 1..24: mol_type = genomic DNA
caatgtccatattgtccactacca

SEQ ID NO: 17 - DNA, 22 bp, Porphyromonas gingivalis
  source 1..22: mol_type = genomic DNA
ctgggcaaagcgtctggaaaac

SEQ ID NO: 18 - DNA, 24 bp, Porphyromonas gingivalis
  source 1..24: mol_type = genomic DNA
gaaatcacgaatcggggaaatctc

SEQ ID NO: 19 - DNA, 20 bp, synthetic construct
  source 1..20: mol_type = other DNA
gtgstgcayggytgtcgtca

SEQ ID NO: 20 - DNA, 20 bp, synthetic construct
  source 1..20: mol_type = other DNA
acgtcrtccmcaccttcctc